(19) United States
(12) Patent Application Publication
(10) Pub. No.: US 2010/0190175 A1
(43) Pub. Date: Jul. 29, 2010

(54) METHODS OF RANDOM MUTAGENESIS AND METHODS OF MODIFYING NUCLEIC ACIDS USING TRANSLESION DNA POLYMERASES

(75) Inventors: Gary F. Gerard, Frederick, MD (US); Zhihao Qiu, Gaithersburg, MD (US); Martin Anthony Gleeson, San Diego, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/727,200

(22) Filed: Mar. 18, 2010

Related U.S. Application Data
(63) Continuation of application No. 11/736,803, filed on Apr. 18, 2007, now abandoned.
Application No. 11/736,803 is a continuation of application No. 10/345,412, filed on Jan. 16, 2003, now abandoned.
(60) Provisional application No. 60/348,677, filed on Jan. 17, 2002.

(51) Int. Cl. C12Q 1/68; C12P 19/34

(52) U.S. Cl. 435/6; 435/91.2

(57) ABSTRACT

The invention is related generally to methods of amplifying or synthesizing or producing nucleic acid molecules using Translesion DNA polymerases. In particular, the invention relates to methods of introducing a random mutation into a nucleic acid and encoded polypeptide using Translesion DNA polymerases. The invention also relates to methods of introducing a modified nucleotide into a nucleic acid using Translesion DNA polymerases. The invention also relates to mutagenized and modified nucleic acid molecules and proteins produced by these methods, and to fragments or derivatives thereof. The invention also relates to vectors and host cells comprising mutagenized nucleic acid molecules, fragments, or derivatives. The invention also relates to the use of mutagenized nucleic acid molecules to produce desired polypeptides and uses of modified nucleic acid molecules to analyze samples. The invention also relates to kits or compositions or compounds for use in the invention or for carrying out the invention.

### CROSS-REFERENCE TO RELATED APPLICATIONS

This application claims the benefit of U.S. Provisional Application No. 60/348,677, filed Jan. 17, 2002.

### STATEMENT REGARDING FEDERALLY-SPONSORED RESEARCH AND DEVELOPMENT

Not applicable.

### REFERENCE TO MICROFICHE APPENDIX/SEQUENCE LISTING/TABLE/COMPUTER PROGRAM LISTING APPENDIX

### Submitted on a Compact Disc and an Incorporation-by-Reference of the Material on the Compact Disc

Not applicable.

### BACKGROUND OF THE INVENTION

1. Field of the Invention

The present invention is in the fields of molecular biology and protein chemistry. The invention is related generally to methods of synthesizing or amplifying (copying) nucleic acids using one or more Translesion DNA polymerases. In some aspects, the methods are directed to introducing a random mutation into a nucleic acid and/or to introducing a random mutation into an encoded polypeptide. In other aspects, the methods are directed to introducing a modified nucleotide into a nucleic acid. In further aspects, the methods comprise use of at least one Translesion DNA polymerase and, optionally, at least one non-translesion DNA polymerase. The methods also comprise use of at least two Translesion DNA polymerases and optionally, at least one non-translesion DNA polymerase. The invention also relates to mutagenized and/or modified nucleic acid molecules produced by these methods, and to fragments or derivatives thereof. The invention also relates to vectors and host cells comprising such mutagenized and/or modified nucleic acid molecules, fragments, or derivatives. The invention also relates to the use of mutagenized and/or modified nucleic acid molecules to produce desired polypeptides or proteins and to use of the modified nucleic acid molecules to analyze sample nucleic acids, to detect one or more nucleic acid molecules in a sample and/or to determine the amount (exactly or approximately) of one or more nucleic acid molecules in a sample. The invention also relates to kits or compositions or compounds for use in the invention or for carrying out the invention.

2. Related Art

DNA Amplification

In order to increase the copy number of, or “amplify,” specific sequences of DNA in a sample, investigators have relied on a number of amplification techniques. A commonly used amplification technique is the Polymerase Chain Reaction (“PCR”) method described by Mullis and colleagues (U.S. Pat. Nos. 4,683,195; 4,683,202; and 4,800,159). This method uses “primer” sequences which are complementary to opposing regions on the DNA sequence to be amplified. These primers are added to the DNA target sample, along with a molar excess of nucleotide bases and a DNA polymerase (e.g., Tag polymerase), and the primers bind to their target via base-specific binding interactions (i.e., adenine binds to thymine, cytosine to guanine).

If the target polynucleotide contains two strands, it may be necessary to separate the strands of the nucleic acid before it can be used as the template, either as a separate step or simultaneously with the synthesis of the primer extension products. This strand separation can be accomplished by any suitable denaturing method including physical, chemical or enzymatic means. One physical method of separating the strands of the polynucleotide involves heating the polynucleotide until it is substantially denatured. Strand separation may also be induced by an enzyme from the class of enzymes known as helicases or the enzyme RecA, which has helicase activity and in the presence of rATP is known to denature DNA. The reaction conditions suitable for separating the strands of polynucleotides with helicases are described by Cold Spring Harbor Symposia on Quantitative Biology, Vol. XLIII “DNA: Replication and Recombination” (New York: Cold Spring Harbor Laboratory, 1978), B. Kuhn et al., “DNA Helicases”, pp. 63-67, and techniques for using RecA are reviewed in C. Radding, Ann. Rev. Genetics, 16:405-37 (1982). Strand separation may also be performed by applying a voltage (U.S. Pat. No. 6,197,508).

Other techniques for amplification of target nucleic acid sequences have also been developed. For example, Walker et al. (U.S. Pat. No. 5,455,166; EP 0 684 315) described a method called Strand Displacement Amplification (SDA), which differs from PCR in that it operates at a single temperature and uses a polymerase/endonuclease combination of enzymes to generate single-stranded fragments of the target DNA sequence, which then serve as templates for the production of complementary DNA (cDNA) strands. An alternative amplification procedure, termed Nucleic Acid Sequence-Based Amplification (NASBA) was disclosed by Davey et al. (U.S. Pat. No. 5,409,818; EP 0 329 822). Similar to SDA, NASBA employs an isothermal reaction, but is based on the use of RNA primers for amplification rather than DNA primers as in PCR or SDA. Another known amplification procedure includes Promoter Ligation Activated Transcriptase (LAT) described by Berninger et al. (U.S. Pat. No. 5,194,370). Single primer amplification provides for the amplification of a template that possesses a stem-loop or inverted repeat structure where the template is flanked by relatively short complementary sequences. U.S. Pat. No. 5,066,584 discloses a method wherein single stranded DNA can be generated by the polymerase chain reaction using two oligonucleotide primers, one present in a limiting concentration. U.S. Pat. No. 5,340,728 discloses an improved method for performing a nested polymerase chain reaction (PCR) amplification of a targeted piece of DNA, wherein by controlling the annealing times and concentration of both the outer and the inner set of primers according to the method disclosed, highly specific and efficient amplification of a targeted piece of DNA can be achieved without depletion or removal of the outer primers from the reaction mixture vessel. U.S. Pat. No. 5,286,632 discloses recombination PCR(RPCR) wherein PCR is used with at least two primer species to add double-stranded homologous ends to DNA such that the homologous ends undergo in vivo recombination following transfection of host cells.

Horton et al. (1989) Gene 77:61, discloses a method for making chimeric genes using PCR to generate overlapping homologous regions. Silver and Keerikatte (1989) J. Virol. 63:1924 describe another variation of the standard PCR approach (which requires oligonucleotide primers complementary to both ends of the segment to be amplified) to allow amplification of DNA flanked on only one side by a region of known DNA sequence. Triglia et al. (1988) Nucl. Acids Res. 16:8186, describe an approach which requires the inversion of the sequence of interest by circularization and re-opening at a site distinct from the one of interest, and is called “inverted PCR.” U.S. Pat. No. 5,928,905 discloses end-complementary amplification.

### Random Mutagenesis

Random mutagenesis is used to introduce random changes into polynucleotides and encoded proteins (Miller et al., (1992) A Short Course in Bacterial Genetics, CSHL Press, Cold Spring Harbor, N.Y.; and Greener et al., (1994) Strategies in Mol Biol 7:32-34) and is used in directed evolution strategies. Random mutagenesis and other directed evolution strategies have advantages over rational design methods by, for example, allowing one to change or optimize a biological molecule in contexts not found in nature. Random mutagenesis is also used in structure-function studies and has the advantage over reverse genetic techniques of allowing one to carry out structure-function studies without making assumptions regarding which regions of a molecule may be essential or dispensable to a particular activity. Further, random mutagenesis can potentially greatly reduce the time and effort needed to generate a large number of progeny for either directed evolution or structure-function studies over current techniques.

More recent methods of random mutagenesis rely on error-prone DNA polymerases. Using these polymerases, randomized (mutagenized) DNA is produced and cloned into expression vectors, and the resulting mutant libraries are screened for activity such as enzymatic or binding activity. The level of desired mutation frequency varies with the application. For example, to analyze protein structure-function relationships, one amino acid change per gene is desired (1-2 base changes per 1000 nucleotides). In directed evolution strategies, mutation frequencies of 1-4 amino acid changes per gene (2-7 nucleotide changes) are desired (Wan, L., et al., Proc. Natl. Acad. Sci. USA 95:12825-12831 (1998); Chemy, J. R., et al., Nature Biotechnology 17:379-384 (1999)). Some strategies involve highly mutagenized libraries containing 20 point mutations per gene (Daugherty, P. S., et al., Proc. Natl. Acad. Sci. USA 97:2029-2034 (2000)).

Up to the present, DNA polymerases were not available with mutation frequencies high enough to generate the required number of mutations per gene during a single round of copying a gene. Protocols were developed to force misincorporation by the use of nucleotide concentration imbalance during a single round of DNA synthesis (Liao, X. and Wise J. A., Gene 88:107-111 (1990)), but the rate of mutation and distribution of mutation type were difficult to control. To address the issues of introducing a sufficiently high number of mutations in a gene while maintaining some control over the number of mutations actually introduced, PCR random mutagenesis with pol Taq was developed (Leung, D. W., et al., Technique 1:11-15 (1989); Cadwell, R. C. and Joyce, G. F., PCR Methods Applications 2:28-33 (1992); Cadwell, R. C. and Joyce, G. F., Mutagenic PCR, in PCR Primer, A Laboratory Manual, C. W. Dieffenbach and G. S. Dveksler (eds.), CSHL Press, pp. 583-589 (1995); Eckert, K. A. and Kunkel, T. A., PCR Methods Applications 1:17-24 (1991); Zhou, Y., et al., Nuc. Acids Res. 19:6052 (1991); Vartanian, J. P., et al., Nuc. Acids Res. 14:2627-2631 (1996); Fromant, M., et al., Anal. Biochem. 224:347-353 (1995); Tindall, K. R. and Kunkel, T. A., Biochem. 27:6008-6013 (1988); Eckert, K. A. and Kunkel, T. A., Nuc. Acids Res. 18:3739-3744 (1990); Huang, M. M., et al., Nuc. Acids Res. 20:4567-4573 (1992)).

The formula f=ne/2 describes the average mutation frequency (f) for PCR amplification as a function of the polymerase error rate per nucleotide per cycle (e) and the number of cycles (n), assuming e is constant at each cycle and the polymerase makes one pass of each DNA molecule per cycle (p=pass number) (Eckert, K. A. and Kunkel, T. A., PCR Methods Applications 1:17-24 (1991)). The frequency of DNA mutations can be controlled by altering the number of cycles (n) and/or the polymerase error rate per nucleotide incorporated (e). It is a given that the number of PCR cycles (n) is greater than or equal to the number passes (p) per cycle, n≧p. As the starting DNA amount is increased, the inequality between n and p increases. That is as the amount of starting DNA increases, the number of DNA molecules in a population that do not get copied during one PCR cycle also increases. So a third variable, starting DNA amount, can be used to influence p and thus the frequency of DNA mutations.

Pol Taq has an average error rate (e) of 1×10⁻⁴ (Table 1; Tindall, K. R. and Kunkel, T. A., Biochem. 27:6008-6013 (1988); Eckert, K. A. and Kunkel, T. A., Nuc. Acids Res. 18:3739-3744 (1990)) so that after n=20 cycles with a single pass per cycle (p=1) there would be on the average one base change in 1000 nucleotides incorporated. But the average error rate of pol Taq does not reflect its misincorporation bias, a strong tendency to misincorporate G whenever a template T is encountered (Table 1 and 3, Tindall, K. R. and Kunkel, T. A., Biochem. 27:6008-6013 (1988); Eckert, K. A. and Kunkel, T. A., Nuc. Acids Res. 18:3739-3744 (1990)). A library of mutants generated with pol Taq using standard PCR reaction conditions will contain predominantly transition mutations and particularly T→C (Zhou, Y., et al., Nuc. Acids Res. 19:6052 (1991)). In addition, many applications require a higher mutation frequency (2-20 base changes per 1000 nucleotides).

To overcome these limitations, protocols were developed that increase the error rate of pol Taq and decrease its misincorporation bias (Leung, D. W., et al., Technique 1:11-15 (1989); Cadwell, R. C. and Joyce, G. F., PCR Methods Applications 2:28-33 (1992); Cadwell, R. C. and Joyce, G. F., Mutagenic PCR, in PCR Primer, A Laboratory Manual, C. W. Dieffenbach and G. S. Dveksler (eds.), CSHL Press, pp. 583-589 (1995); Vartanian, J. P., et al., Nuc. Acids Res. 14:2627-2631 (1996); Fromant, M., et al., Anal. Biochem. 224:347-353 (1995)). Error rate is increased by increasing the Mg++ concentration and by adding the mutagenic divalent metal ion Mn++. Misincorporation bias is reduced by manipulating the relative dNTP concentrations. However, because of the extreme sensitivity of pol Taq to changes in dNTP and Mn++ concentrations, the mutation number and type obtained in a mutant population are often not predictable or reproducible. Unbalancing the dNTP concentrations does not totally eliminate the misincorporation bias of pol Taq (Cadwell, R. C. and Joyce, G. F., Mutagenic PCR, in PCR Primer, A Laboratory Manual, C. W. Dieffenbach and G. S. Dveksler (eds.), CSHL Press, pp. 583-589 (1995)). The modified PCR reaction conditions required frequently produce poor product yields and amplification artifacts (Id.).

At least two companies now offer random mutagenesis systems, Clontech and Stratagene. Clontech sells a system called Diversify PCR Random Mutagnesis Kit. Clontech's kit relies upon the use of Mn++ and nucleotide imbalance to control the mutation frequency and bias of pol Tag. This system suffers from the disadvantages already mentioned in trying to control the mutation frequency and mutation bias of pol Taq. An interesting positive feature of Clontech's kit is the inclusion of a rapid control reaction that allows the relative comparison of mutation rates in the control DNA fragment in two hours following PCR.

Stratagene sells a system called GeneMorph PCR Mutagenesis Kit. Stratagene has taken a different approach in their system. Rather than manipulating the error frequency of pol Taq, they manipulate the starting DNA concentration over 5 logs in PCR performed under one set of reaction conditions. This influences the number of mutations introduced in the final amplified DNA population as already discussed. They have also introduced the use of a new thermal stable DNA polymerase, Mutazyme™, that has an error rate 5-10 times greater than pol Tag. This system suffers from the unpredictability of the number of mutations actually produced with a new DNA template at a selected concentration, and from the mutation pattern bias of Mutazyme™.

### Incorporation of Modified Nucleotides

Numerous methods and systems have been developed for the detection, quantitation, and analysis of polynucleotides in drug development, diagnostics, and research. These methods are used in disease diagnosis, for example by detecting polynucleotides of infectious organisms or detecting somatic and heritable mutations, and in basic and industrial research, for example by analyzing gene expression.

An expanding area of polynucleotide analysis is DNA array technology. This technology using arrays of nucleic acid probes, such as oligonucleotides, to detect complementary nucleic acid sequences in a sample nucleic acid of interest (the “target” nucleic acid). For example, an array of nucleic acid probes is fabricated at known locations on a substrate such as a chip. A labeled nucleic acid is then brought into contact with the chip and a scanner generates an image file indicating the locations where the labeled nucleic acids are bound to the chip. Based upon the image file and identities of the probes at specific locations, it becomes possible to extract information such as the expression pattern of a nucleic acid of interest (see, e.g., U.S. Pat. No. 6,225,077).

Methods using arrays of nucleic acids immobilized on a solid substrate are disclosed, for example, in U.S. Pat. No. 5,510,270. In this method, an array of diverse nucleic acids is formed on a substrate. The fabrication of arrays of polymers, such as nucleic acids, on a solid substrate, and methods of use of the arrays in different assays, are described in: U.S. Pat. Nos. 6,203,989, 6,200,757, 6,180,351, 6,156,501, 6,083,726, 5,981,185, 5,744,101, 5,677,195, 5,624,711, 5,599,695, 5,445,934, 5,384,261, 5,571,639, 5,451,683, 5,424,186, 5,412,087, 5,384,261, 5,252,743 and 5,143,854; PCT WO 92/10092; PCT WO 93/09668; PCT WO 97/10365. Improved methods for minimizing the effects of random or systematic errors in array technology are disclosed in U.S. Pat. No. 6,223,127.

Accessing genetic information using high density DNA arrays is further described in Chee, Science 274:610-614 (1996). The combination of photolithographic and fabrication techniques allows each probe sequence to occupy a very small site on the support. The site may be as small as a few microns or even a small molecule. Such probe arrays may be of the type known as Very Large Scale Immobilized Polymer Synthesis (VLSIPS™). U.S. Pat. Nos. 5,631,734 and 5,143,854 and PCT patent publication Nos. WO 90/15070 and 92/10092.

Typically, the existence of a nucleic acid of interest in array technology and other DNA detection methods is indicated by the presence or absence of an observable “label” attached to a probe or attached to amplified sample DNA. A convenient method for incorporating a label or other modification into DNA would be to use in vitro amplification of a nucleic acid template using DNA polymerase. However, commercially available DNA polymerases are inefficient at incorporating modified nucleotides, particularly ones with bulky groups. Accordingly, there exists a need for more efficient incorporation of modified nucleotides, particularly labeled nucleotides, during amplification or synthesis of a nucleic acid template. Efficient incorporation of such nucleotides will allow for improved synthesis of labeled probes which may be used in the research market as well as in the field of diagnostics.

### Translesion DNA Polymerases

In the past few years a new superfamily of DNA polymerases has been discovered whose members function in the replication of damaged DNA (Goodman, M., TIBS 25:189-195 (2000); Hubscher, U., et al., TIBS 25:143-147 (2000); Goodman, M. F. and Tippin, B., Curr. Opin. Genetics & Dev. 10:162-168 (2000); Woodgate, R., Genes & Dev. 13:2191-2195 (1999); Friedberg, E. C. and Gerlach, U. L., Cell 98:413-416 (1999); Johnson, R. E., et al., Proc. Natl. Acad. Sci. USA 96:12224-12226 (1999); Baynton, K. and Fuchs, R. P. P., TIBS 25:74-79 (2000); Friedberg, E. C., et al., Proc. Natl. Acad. Sci. USA 97:5681-5683 (2000); Zhang, Y., et al., Mol. Cell. Biol. 20:7099-7108 (2000); McDonald, J. P., et al., Philos. Trans. R. Soc. Lond. B. Biol. Sci. 356:53-60 (2001)). The superfamily is called UmuC/DinB/Rad30/Rev 1 after the four prototypic genes that define the subfamilies within this superfamily (see below). This superfamily will be referred to herein as the Translesion Superfamily of DNA polymerases, and includes E. coli pol IV and pol V, and eukaryotic pol (zeta), η(eta), t (iota), δ (kappa), and θ (theta).

Previously identified DNA polymerase superfamilies include the A, B, C, and X Superfamilies. These superfamilies include, for example, (A) E. coli pol 1, pol T7, pol T5, pol Tag, pol Tth, pol Tne, reverse transcriptases, and eukaryotic pol γ (gamma); (B) E. coli pol II, eukaryotic pol a (alpha), eukaryotic δ (delta), eukaryotic ε (epsilon), pol T4, pol (1329, pol Pfu, and pol KOD (Pfx); (C) E. coli pol III α subunit; and (X) eukaryotic pol β (beta), eukaryotic λ (lambda), eukaryotic μ (mu), and TdT.

Pol III holoenzyme is a member of the C Superfamily of DNA polymerases. It represents the typical genome replicative DNA polymerase with high fidelity (exo+; contains proofreading 3′→5′ exonuclease activity), high processivity (once bound to a template-primer it remains bound through many polymerization events), and minimal ability to bypass lesions in DNA.

The Translesion Superfamily members have several unusual characteristics that set them apart from other DNA polymerases. For example, these DNA polymerases are highly error prone (Table 1). A typical replicative DNA polymerase, such as E. coli pol III holoenzyme, has an error rate (mutations introduced/nucleotide incorporated) of about 5×10⁻⁶ (Matsuda, T. et al. Nature 404: 1011-1013 (2000)). Enzymes previously thought to be error prone include two retroviral reverse transcriptases (RTs) and pol Taq, whose error rates are 0.5-1×10⁻⁴. Notably, members of the Translesion Superfamily, pol κ (kappa) and pol η (eta) in particular, have error rates of 2-4×10⁻² (Table 1). Thus, they make an error once in every 25 to 50 nucleotides incorporated. A third member, pol t, actually violates Watson-Crick base-pairing rules in its nucleotide incorporation preferences (Table 2).

TABLE 1
Base Substitution Mutation Frequencies of DNA Polymerases

| | | | Mutation Frequency/Nucleotide Incorporated × 10⁻⁶ | | | | | | |
|---|---|---|---|---|---|---|---|---|---|
| Mutation | | | Pol | Pol | hPol | M-MLV | AMV | hPol | Pol |
| T/N^{a} | Transition | Transversion | III^{b,c} | V^{c,d} | κ^{e,f} | RT^{e,g} | RT^{e,h} | η^{e,i} | Taq^{e,j} |
| G/T | G→A | | 0.3 | 5 | 1,500 | 9 | 6 | 3,200 | 7 |
| T/G | T→C | | 0.2 | 42 | 2,400 | 6 | 7 | 13,700 | 62 |
| A/C | A→G | | 0.2 | 3 | 800 | 5 | 31 | 2,600 | 3.5 |
| C/A | C→T | | 1.7 | 13 | 1,000 | 1 | 0 | 2,900 | 7 |
| G/G | | G→C | 0.8 | 13 | 1,000 | 4 | 0 | 600 | 7 |
| G/A | | G→T | 0.3 | 3 | 1,000 | 4 | 0.7 | 1,100 | 7 |
| T/T | | T→A | 0.3 | 26 | 650 | 9 | 7 | 2,000 | 10 |
| T/C | | T→G | 0.2 | 8 | 6,800 | 9 | 7 | 1,200 | 0 |
| A/A | | A→T | 0.2 | 48 | 300 | 6 | 6 | 3,900 | 0 |
| A/G | | A→C | 0.3 | 19 | 100 | 3 | 4 | 3,200 | 3.5 |
| C/C | | C→G | 0.2 | 3 | 300 | 1 | 2 | 0 | 3.5 |
| C/T | | C→A | 0.2 | 11 | 1,400 | 0 | 2 | 750 | 0 |

| | | | | | | | |
|---|---|---|---|---|---|---|---|
| Overall Mutation | 4.9 | 194 | 17,210 | 55 | 74 | 35,150 | 111 |
| Frequency × 10⁻⁶ | | | | | | | |

^{a}T/N, template nucleotide and dNTP incorporated
^{b}Pol III holoenzyme
^{c}Replication of the cro gene was used to determine mutation frequencies
^{d}Pol V mut minus β, γ complex
^{e}Replication of the LacZα gene was used to determine mutation frequencies
^{f}87% of the mutations sequenced were point mutations, and 13% were deletions or insertions
^{g}46% of the mutations sequenced were point mutations, and 54% were deletions or insertions
^{h}72% of the mutations sequenced were point mutations, and 28% were deletions or insertions
ⁱ81% of the mutations sequenced were point mutations, and 19% were deletions, insertions, or tandem double-base substitutions
^{j}80% of the mutations sequenced were point mutations, and 20% were deletions or insertions

TABLE 2
Mispair Formation Rates of DNA Polymerases × 10^{-4a}

| | | | Pol | pol | pol | pol | pol | pol | pol | pol | pol |
|---|---|---|---|---|---|---|---|---|---|---|---|
| T/N^{b} | Transition | Transversion | III^{c} | IV^{d} | V^{f} | θ^{g} | κ^{h} | ιⁱ | ζ^{j} | η^{k} | η^{l} |
| G/T | G→A | | 4.8 | 8.5 | 48 | 2.2 | 22 | 380 | 1.1 | 44 | 29 |
| T/G | T→C | | 0.3 | 3.6 | 24 | 4.4 | 230 | 100,000 | 41 | 53 | 110 |
| A/C | A→G | | 1.6 | 1.0 | 6 | 8 | 13 | 0.01 | 1.1 | 33 | 31 |
| C/A | C→T | | 0.7 | 1.3 | 5 | 2.8 | 450 | 420 | 0.5 | 58 | 11 |
| G/G | | G→C | 0.3 | 17^{e} | 27 | 2.2 | 13 | 46 | 1.4 | 3.8 | 48 |
| G/A | | G→T | 1.0 | 6.7 | 13 | 8.9 | 8 | 88 | 0.7 | 3.1 | 88 |
| T/T | | T→A | 1.2 | 0.9 | 37 | 1.5 | 44 | 54,000 | 0.5 | 88 | 94 |
| T/C | | T→G | 0.5 | 2.2 | 8 | 2.9 | 140 | 3,000 | 0.2 | 65 | 83 |
| A/A | | A→T | 0.1 | 0.5 | 0.7 | 6 | 5.2 | 3.4 | 2.5 | 87 | 96 |
| A/G | | A→C | 0.3 | 1.5 | 3 | 30 | 24 | 2 | 13 | 26 | 32 |
| C/C | | C→G | <0.1 | 0.4 | <0.1 | 9.4 | 11 | 230 | 0.4 | 230 | 34 |
| C/T | | C→A | <0.1 | 1.4 | 7 | 1.9 | 580 | 700 | 0.5 | 32 | 12 |

^{a}The ratio of the efficiency of incorporation of incorrect versus correct nucleotide
^{b}T/N, template nucleotide and dNTP incorporated
^{c}Pol III (α + β, γ complex + SSB, no ε)
^{d}Pol IV (β, γ complex + SSB)
^{e}High rate due to dNTP-stabilized misalignment where G is incorporated opposite a template C immediately
downstream from a template G (pol IV has a propensity to catalyze B1 frameshift errors).
^{f}Pol V mut;
^{g}Human pol θ;
^{h}Human pol κ;
ⁱHuman pol ι;
^{j}Human pol ζ,;
^{k}Yeast pol η;
^{l}Human pol η

Members of the Translesion Superfamily of DNA polymerases have several additional properties of interest. First, they are nonprocessive; that is, they dissociate from template-primer after almost every nucleotide incorporation event. Second, the mutation frequency spectra of the Translesion enzymes, particularly pol κ and pol η, are much more uniform than that of pol Taq, the DNA polymerase presently used to generate random mutations (Table 3). Therefore, mutations introduced by pol κ or pol η, for example, will have a much-reduced bias towards a particular type. Third, they also lack proofreading 3′→5′ exonuclease activity.

TABLE 3
Distribution Pattern of Mutation Frequencies of DNA Polymerases^{a}

| | | | Relative Mutation Frequency | | | | | | |
|---|---|---|---|---|---|---|---|---|---|
| Mutation | | | Pol | Pol | hPol | M-MLV | AMV | hPol | Pol |
| T/N^{b} | Transition | Transversion | III | IV | κ | RT | RT | η | Taq |
| G/T | G→A | | 1.5 | 1.7 | 15 | 9 | 6 | 5.3 | 2 |
| T/G | T→C | | 1 | 14 | 24 | 6 | 7 | 22.8 | 17.7 |
| A/C | A→G | | 1 | 1 | 8 | 5 | 31 | 4.3 | 1 |
| C/A | C→T | | 8.5 | 4.3 | 10 | 1 | 0 | 4.8 | 2 |
| G/G | | G→C | 4 | 4.3 | 10 | 4 | 0 | 1 | 2 |
| G/A | | G→T | 1.5 | 1 | 10 | 4 | 1 | 1.8 | 2 |
| T/T | | T→A | 1.5 | 8.7 | 6.5 | 9 | 7 | 3.3 | 1 |
| T/C | | T→G | 1 | 2.7 | 68 | 9 | 7 | 2.0 | 0 |
| A/A | | A→T | 1 | 16 | 3 | 6 | 6 | 6.5 | 0 |
| A/G | | A→C | 1.5 | 6.3 | 1 | 3 | 4 | 5.3 | 1 |
| C/C | | C→G | 1 | 1 | 3 | 1 | 2 | 0 | 1 |
| CYT | | C→A | 1 | 3.7 | 14 | 0 | 2 | 1.3 | 0 |

^{a}For each DNA polymerase, values were derived from the mutation frequencies in Table 2 by setting the lowest frequency value above 0 at 1 and calculating a ratio to the remaining values
^{b}T/N, template nucleotide and dNTP incorporated

### Subfamilies of Translesion DNA Polymerases

1. The E. coli UmuC (Pol V) Subfamily. (See, e.g., Bruck, I., et al., J. Biol. Chem. 271:10767-10774 (1996); Tang, M., et al., Proc. Natl. Acad. Sci. USA 95:9755-9760 (1998); Tang, M., et al., Proc. Natl. Acad. Sci. USA 96:8919-8924 (1999); Reuven, N. B., et al., J. Biol. Chem. 274:31763-31766 (1999); Maor-Shoshani, A., et al., Proc. Natl. Acad. Sci. USA 97:565-570 (2000); Tang, M., et al., Nature 404:1014-1018 (2000); Pham, P., et al., Nature 409:366-370 (2001).)

Pol V is a complex of the E. coli UmuC gene product (catalytic subunit of 422 aa) with two subunits derived from the UmuD gene product cleaved with RecA: UmuD′₂C (pol V) (Tang, M., et al., Proc. Natl. Acad. Sci. USA 96:8919-8924 (1999); Reuven, N. B., et al., J. Biol. Chem. 274:31763-31766 (1999); Maor-Shoshani, A., et al., Proc. Natl. Acad. Sci. USA 97:565-570 (2000); Tang, M., et al., Nature 404:1014-1018 (2000); Pham, P., et al., Nature 409:366-370 (2001)).

Pol V has no 3′→5′ exonuclease proofreading activity (Tang, M., et al., Nature 404:1014-1018 (2000)). Pol V has low processivity, dissociating after incorporation of 6 to 8 nucleotides under the best of conditions and is distributive in the absence of accessory proteins (Tang, M., et al., Nature 404:1014-1018 (2000)).

Pol V requires RecA*, β processivity clamp, γ clamp-loading complex (5 proteins), and ssb to carry out efficient copying of DNA (Pham, P., et al., Nature 409:366-370 (2001)). This complex of proteins is called a mutasome or Pol V mut (UmuD′₂C/RecA*/(β,γ complex/ssb). Pol V mut has a relatively high rate of base mispair formation when copying DNA with rates of 10⁻³ to 10⁴ (Tang, M., et al., Nature 404:1014-1018 (2000)) (Table 1). In copying DNA with Pol V, it appears that ATPγ-S can be substituted for β, γ complex (Pham, P., et al., Nature 409: 366-370 (2001). There is no data available on whether the combination of just Pol V, ssb, and ATPγ-S could be used to copy DNA efficiently.

2. The E. coli DinB (Pol IV), human DinB1 (Pol κ or Pol θ) Subfamily. (See, e.g., Tang, M., et al., Nature 404:1014-1018 (2000); Wagner, J., et al., Mol. Cell. 4:281-286 (1999); Wagner, J. and Nohmi, T., J. Bacteriol. 182:4587-4595 (1999); Gerlach, V. L., et al., Proc. Natl. Acad. Sci. USA 96:11922-11927 (1999); Gerlach, V. L., et al., J. Biol. Chem. 276:92-98 (2001); Zhang, Y., et al., Nuc. Acids Res. 28:4138-4146 (2000); Zhang, Y., et al., Nuc. Acids Res. 28:4147-4156 (2000); Johnson, R. E., Proc. Natl. Acad. Sci. USA 97:3838-3843 (2000); Ohashi, E., et al., Gen. Dev. 14:1589-1594 (2000); Ohashi, E., et al., J. Biol. Chem. 275:39678-39684 (2000).)

Pol IV is the gene product of the E. coli DinB gene (351 aa) (Tang, M., et al., Nature 404:1014-1018 (2000); Wagner, J., et al., Mol. Cell. 4:281-286 (1999); Wagner, J. and Nohmi, T., J. Bacteriol. 182:4587-4595 (1999)). Pol IV has no 3′→5′ exonuclease proofreading activity (Tang, M., et al., Nature 404:1014-1018 (2000); Wagner, J., et al., Mol. Cell. 4:281-286 (1999)). It has low processivity (dissociates after 6 to 8 nucleotides) under the best of conditions (in the presence of accessory factors) (Tang, M., et al., Nature 404:1014-1018 (2000)), and is distributive in the absence of accessory proteins (Wagner, J., et al., Mol. Cell. 4:281-286 (1999)).

The copying efficiency of Pol IV is increased dramatically by ssb and β,γ complex (particularly β,γ complex) (Tang, M., et al., Nature 404:1014-1018 (2000)).

Pol IV is less error prone than pol V mut when copying DNA with mispair formation rates of 10⁻⁴ to 10⁻⁵ (Tang, M., et al., Nature 404:1014-1018 (2000)) (Table 1). Pol IV is prone to elongate bulged (misaligned) template-primer (Wagner, J., et al., Mol. Cell. 4:281-286 (1999)), resulting in single-base deletions in DNA products (Wagner, J. and Nohmi, T., J. Bacteriol. 182:4587-4595 (1999)). Pol IV base substitution errors are biased towards a G substitution for another base and most often occur at the sequence 5′-GX-3′ where X represents the base (T, A, or C) that is mutated to G (Tang, M., et al., Nature 404:1014-1018 (2000); Wagner, J. and Nohmi, T., J. Bacteriol. 182:4587-4595 (1999)).

Pol κ (Gerlach, V. L., et al., Proc. Natl. Acad. Sci. USA 96:11922-11927 (1999); Gerlach, V. L., et al., J. Biol. Chem. 276:92-98 (2001); Zhang, Y., et al., Nuc. Acids Res. 28:4138-4146 (2000); Zhang, Y., et al., Nuc. Acids Res. 28:4147-4156 (2000); Ohashi, E., et al., Gen. Dev. 14:1589-1594 (2000); Ohashi, E., et al., J. Biol. Chem. 275:39678-39684 (2000)) (also called pol θ, Johnson, R. E., Proc. Natl. Acad. Sci. USA 97:3838-3843 (2000)) is the gene product of the human and mouse DinB1 gene (870 aa; 99 KDa) (Gerlach, V. L., et al., Proc. Natl. Acad. Sci. USA 96:11922-11927 (1999); Gerlach, V. L., et al., J. Biol. Chem. 276:92-98 (2001); Zhang, Y., et al., Nuc. Acids Res. 28:4138-4146 (2000); Zhang, Y., et al., Nuc. Acids Res. 28:4147-4156 (2000) Ohashi, E., et al., Gen. Dev. 14:1589-1594 (2000); Ohashi, E., et al., J. Biol. Chem. 275:39678-39684 (2000)).

Pol κ has no 3′→5′ exonuclease proofreading activity (Gerlach, V. L., et al., J. Biol. Chem. 276:92-98 (2001); Ohashi, E., et al., Gen. Dev. 14:1589-1594 (2000)). The processivity of full-length pol lc is moderate (˜25 nt) (Gerlach, V. L., et al., J. Biol. Chem. 276:92-98 (2001); Ohashi, E., et al., J. Biol. Chem. 275:39678-39684 (2000)), and the processivity of a C-terminal truncated pol in which a putative DNA binding domain has been deleted is low (Ohashi, E., et al., Gen. Dev. 14:1589-1594 (2000); Ohashi, E., et al., J. Biol. Chem. 275:39678-39684 (2000)).

Addition of human PCNA (the human sliding clamp analogous to E. coli β,γ-complex for maintaining processivity of pol 5 during chain elongation) did not increase the processivity of pol κ on undamaged DNA templates (Gerlach, V. L., et al., J. Biol. Chem. 276:92-98 (2001)). The effects of RP-A (ssb) and PCNA together have not been determined.

Like E. coli pol IV, human pol K can prime synthesis from a misaligned (bulged) template-primer (Gerlach, V. L., et al., J. Biol. Chem. 276:92-98 (2001); Zhang, Y., et al., Nuc. Acids Res. 28:4138-4146 (2000)). The error rate of pol on undamaged DNA templates is 5×10⁻³ or one error for every 200 nucleotides synthesized (Zhang, Y., et al., Nuc. Acids Res. 28:4138-4146 (2000); Ohashi, E., et al., J. Biol. Chem. 275:39678-39684 (2000)) (Table 2). Most of these errors (64-90%) are single-base misinsertions and not deletions or insertions (Zhang, Y., et al., Nuc. Acids Res. 28:4138-4146 (2000); Ohashi, E., et al., J. Biol. Chem. 275:39678-39684 (2000)).

3. The yeast REV1/REV3/REV7 (dCTP Transferase, eukaryotic Pol ζ) Subfamily. (See, e.g., Shibutani, S., et al., Nature 349:431-434 (1991); Nelson, J. R., et al., Science 272:1646-1649 (1996); Nelson J. R., et al., Nature 382:729-731 (1996); Gibbs, P. E. M., et al., Proc. Natl. Acad. Sci. USA 95:6876-6880 (1998); Johnson, R. E., et al., Nature 406:1015-1019 (2000); Benmark, M., et al., Curr. Biol. 10:1213-1216 (2000); Kawamura, K., et al., Int. J. Oncol. 18:97-103 (2001); Lawrence, C. W. and Maher, V. M., Philos. Trans. R. Soc. Lond. B. Biol. Sci. 356:41-46 (2001); Murakumo, Y., et al., J. Biol. Chem. 275:4391-4397 (2000); Baynton, K., et al., Mol. Cell. Biol. 18:960-966 (1998); Baynton, K., et al., Mol. Microbiol. 34:124-133 (1999); Gibbs, P. E. M., Proc. Natl. Acad. Sci. USA 97:4186-4191 (2000); Harfe, B. D. and Jinks-Robertson, S., Mol. Cell. 6:1491-1499 (2000).)

Yeast dCTP transferase is the gene product of the yeast REV 1 gene (985 aa) (Nelson J. R., et al., Nature 382:729-731 (1996)). It incorporates a C opposite an abasic site at the 3′ end of a DNA primer in a template-dependent reaction (Nelson J. R., et al., Nature 382:729-731 (1996)). dCTP transferase does not add nucleotides beyond the C incorporated at an abasic site (Nelson J. R., et al., Nature 382:729-731 (1996)).

Yeast pol ζ is the gene product of the yeast REV3 gene (1,504 aa; catalytic subunit) and REV7 gene (Nelson, J. R., et al., Science 272:1646-1649 (1996)). It has no 3′→5′ exonuclease proofreading activity (Nelson, J. R., et al., Science 272:1646-1649 (1996)) and relatively low processivity (Nelson, J. R., et al., Science 272:1646-1649 (1996)). Pol ζ efficiently synthesizes DNA from most mispaired 3′ ends and the error rate for mispair extension is extraordinarily high at 10⁻¹ to 10⁻² (Johnson, R. E., et al., Nature 406:1015-1019 (2000)). The error rate of pol on undamaged DNA for mispair formation is relatively low at 10⁻⁴ to 10⁻⁵ (Johnson, R. E., et al., Nature 406:1015-1019 (2000)) (Table 2).

4. The yeast RAD30, human RAD30A (Pol η) Subfamily. (See, e.g., Johnson, R. E., et al., Science 283:1001-1004 (1999); Johnson, R. E., et al., J. Biol. Chem. 274:15975-15977 (1999); Washington, M. T., et al., J. Biol. Chem. 274:36835-36838 (1999); Washington, M. T., et al., Proc. Natl. Acad. Sci. USA 97:3094-3099 (2000); Washington, M. T., et al., J. Biol. Chem. 276:2263-2266 (2001); Haracska, L., et al., Nature Genetics 25:458-461 (2000); Yu, S. L., et al., Mol. Cell. Biol. 21:185-188 (2001); Masutani, C., et al., Nature 399:700-704 (1999); Johnson, R. E., et al., Science 285:263-265 (1999); Masutani, C., et al., EMBO J. 18:3491-3501 (1999); McDonald, J. P., et al., Genomics 60:20-30 (1999); Johnson, R. E., et al., J. Biol. Chem. 275:7447-7450 (2000); Matsuda, T., et al., Nature 404:1011-1013 (2000); Bebenek, K., et al., J. Biol. Chem. 276:2317-2320 (2001); Zhang, Y., et al., Nuc. Acids Res. 28:4717-4724 (2000); Yuan, F., et al., J. Biol. Chem. 275:8233-8239 (2000); Haracska, L., et al., J. Biol. Chem. 276:6861-6866 (2001); Minko, I. G., et al., J. Biol. Chem. 276:2517-2522 (2001); Haracska, L., et al., Mol. Cell. Biol. 20:8001-8007 (2000); Masutani, C., et al., The EMBO J. 19:3100-3109 (2000).)

Yeast pot η is the product of the yeast RAD30 gene (632 aa) (Johnson, R. E., et al., Science 283:1001-1004 (1999); Johnson, R. E., et al., J. Biol. Chem. 274:15975-15977 (1999)) and human pot 11 is the product of the human RAD30A gene (713 aa) (Masutani, C., et al., Nature 399:700-704 (1999); Johnson, R. E., et al., Science 285:263-265 (1999); Masutani, C., et al., EMBO J. 18:3491-3501 (1999); McDonald, J. P., et al., Genomics 60:20-30 (1999)).

Pol η has no 3′→5′ exonuclease proofreading activity (Matsuda, T., et al., Nature 404:1011-1013 (2000); Yuan, F., et al., J. Biol. Chem. 275:8233-8239 (2000)) and has low processivity. Most pot η molecules will dissociate after no more than 6 nucleotides are incorporated (Washington, M. T., et al., J. Biol. Chem. 274:36835-36838 (1999); Bebenek, K., et al., J. Biol. Chem. 276:2317-2320 (2001)).

Pol η has a high rate of mispair formation in copying undamaged DNA (10⁻² to 10⁻³) and the mispair frequencies are relatively uniform across the spectrum of possibilities (Washington, M. T., et al., J. Biol. Chem. 274:36835-36838 (1999); Johnson, R. E., et al., J. Biol. Chem. 275:7447-7450 (2000)) (Table 1). The average rate of mispair extension by pol η is less than the rate of mispair formation (10⁻³) (Washington, M. T., et al., J. Biol. Chem. 276:2263-2266 (2001); Bebenek, K., et al., J. Biol. Chem. 276:2317-2320 (2001)). The average error frequency of pol η for copying undamaged DNA for single base substitutions is 1 in 28 nucleotides incorporated (Matsuda, T., et al., Nature 404:1011-1013 (2000)) (Table 2).

5. The human RAD30B (Pol ι) Subfamily. (See, e.g., Johnson, R. E., et al., Nature 406:1015-1019 (2000), Tissier, A., et al., The EMBO J. 19:5259-5266 (2000); Zhang, Y., et al., Mol. Cell. Biol. 20:7099-7108 (2000); Zhang, Y., et al., Nuc. Acids Res. 29:928-935 (2001); Tissier, A., et al., Gen. Dev. 14:1642-1650 (2000).)

Human pol ι is the gene product of the human RAD30B gene (715 aa; 81 Kda) (Johnson, R. E., et al., Nature 406:1015-1019 (2000)). Pol ι has no 3′→5′ exonuclease proofreading activity (Johnson, R. E., et al., Nature 406:1015-1019 (2000); Zhang, Y., et al., Mol. Cell. Biol. 20:7099-7108 (2000)). It appears to be nonprocessive and has difficulty extending primers beyond 6 bases, even with an undamaged DNA template (Johnson, R. E., et al., Nature 406:1015-1019 (2000)). This is due in part to the tendency of pol ι to incorporate a G next to template T more readily than A, and its inability to efficiently extend the T-G mispair (Zhang, Y., et al., Mol. Cell. Biol. 20:7099-7108 (2000)).

Pol ι has extraordinarily high rates of mispair formation at template pyrimidines, T (0.3 to 10) and C (0.02 to 0.07); and lower rates at template purines, G (0.005 to 0.04) and A (0.01×10⁻⁴ to 3×10⁻⁴) (Johnson, R. E., et al., Nature 406:1015-1019 (2000); Zhang, Y., et al., Mol. Cell. Biol. 20:7099-7108 (2000); Tissier, A., et al., Gen. Dev. 14:1642-1650 (2000)) (Table 2).

### SUMMARY OF THE INVENTION

The present invention provides kits, compositions and methods useful in overcoming limitations in random mutagenesis and incorporation of modified nucleotides. The methods of the present invention relate generally to methods of synthesizing or amplifying nucleic acid molecules using one or more Translesion DNA polymerases.

In one aspect, the invention relates to kits and methods and compostions for incorporating random mutations or changes (preferably randomly) in DNA molecules. In this aspect, one or more template nucleic acid molecules and at least one Translesion DNA polymerase are incubated under conditions sufficient to allow synthesis of a complementary nucleic acid molecule (which may be complementary to all or a portion of said one or more of said templates). Such conditions generally require at least one primer and one or more nucleotides (e.g., dNTPs), and may also require buffers, salts and/or accessory proteins. A Translesion DNA polymerase incorporates at least one mutation (which may be one or more deletions, substitutions and insertions or combinations thereof of one or more nucleotides) in the complementary nucleic acid molecule. One or more rounds of synthesis may be performed to incorporate any number of such mutations which are preferably random mutations. One or more non-translesion DNA polymerases may also be used in the present methods. The resulting complementary nucleic acid molecules (mutagenized nucleic acid molecules) may be further amplified using standard amplification techniques such as PCR. More than one Translesion DNA polymerase (which may be the same or different) and more than one non-translesion DNA polymerase (which may be the same or different) may be used. Such polymerases may be mesophilic or thermophilic.

In a preferred aspect, one or more mismatch nucleotides are added to the nucleic acid molecule made by the methods of the invention to produce one or a population of randomly mutagenized nucleic acid molecules and such mutagenized nucleic acid molecules may be used to produce one or a population of polypeptides or proteins having any number of changes in amino acid sequences. Preferably, one or more amino acid substitutions are created in such polypeptides, although other types of changes or combination or changes in amino acid sequence can take place including one or more deletion of amino acids, and one or more insertions of amino acids. Thus, the invention provides methods and requests capable of producing one or more and preferably populations of mutagenized nucleic acid molecules (which may comprise any number of substitution, insertion and/or deletion mutations) and such nucleic acid molecules may be used to produce mutagenized polypeptides or proteins. Such proteins or populations of proteins may then be analyzed for desired functional or activity changes using well known techniques and functional or activity assays. Proteins or polypeptides encoded by the nucleic acid molecules of the invention may be produced by expression of the nucleic acid molecules in a host cell or by using in vitro transcription/translation systems known in the art.

The invention further provides mutagenized nucleic acids produced by the above-described methods and host cells comprising mutagenized nucleic acids of the invention. Such mutagenized nucleic acid molecules may be single or double stranded. Mutagenized nucleic acids are useful for structure-function studies and for optimizing encoded mRNA and polypeptides. Such molecules, especially polypeptides, can be assayed for improved enzymatic activities, receptor properties, ligand interactions, antibiotic or antiviral properties, vaccine efficacy, or antibody binding affinity. The invention also provides polypeptides encoded by the mutagenized nucleic acids of the invention.

In another aspect, the present invention relates to kits and methods of synthesizing modified nucleic acid molecules. In this aspect, one or more template nucleic acid molecules, at least one Translesion DNA polymerase, and one or more modified nucleotides (which may be the same or different) are incubated under conditions sufficient to allow synthesis of a complementary nucleic acid molecule (which may be complementary to all or a portion of said one or more of said templates). Such conditions generally require at least one primer and one or more nucleotides (e.g., dNTPs), and may also require buffers, salts and/or accessory proteins. The Translesion DNA polymerase incorporates the one or more (which may be the same or different) modified nucleotides in the complementary nucleic acid molecule. One or more rounds of synthesis may be used. More than one Translesion DNA polymerase and more than one non-translesion DNA polymerase may be used. Such polymerases may be mesophilic or thermophilic.

The invention also provides modified nucleic acid molecules produced according to the above-described methods. Such modified nucleic acid molecules may be single or double stranded and may comprise any number of the same or different modified nucleotides. Modified nucleic acid molecules include labeled nucleic acid molecules and are useful as detection probes. Depending on the modified nucleotide(s) used during synthesis, the modified molecules may contain one or a number of modifications. Where multiple modifications are used, the molecules may comprise a number of the same or different modifications such as labels. Thus, one type or multiple different modified nucleotides may be used during synthesis of nucleic acid molecules to provide for the modified nucleic acid molecules of the invention. Such modified nucleic acid molecules will thus comprise one or more modified nucleotides (which may be the same or different). The invention also provides uses of the modified nucleic acids for analyzing samples.

### BRIEF DESCRIPTION OF DRAWINGS

FIG. 1 is a schematic of the random mutagenesis technique using a mesophilic Translesion DNA polymerase. Increased temperature during amplification may inactivate or partially inactivate Translesion DNA polymerase activity such that introduction of mutations with Translesion DNA polymerase during PCR is limited or eliminated. Use of thermophilic Translesion DNA polymerase during amplification may provide for additional mutagenesis of the nucleic acid molecules.

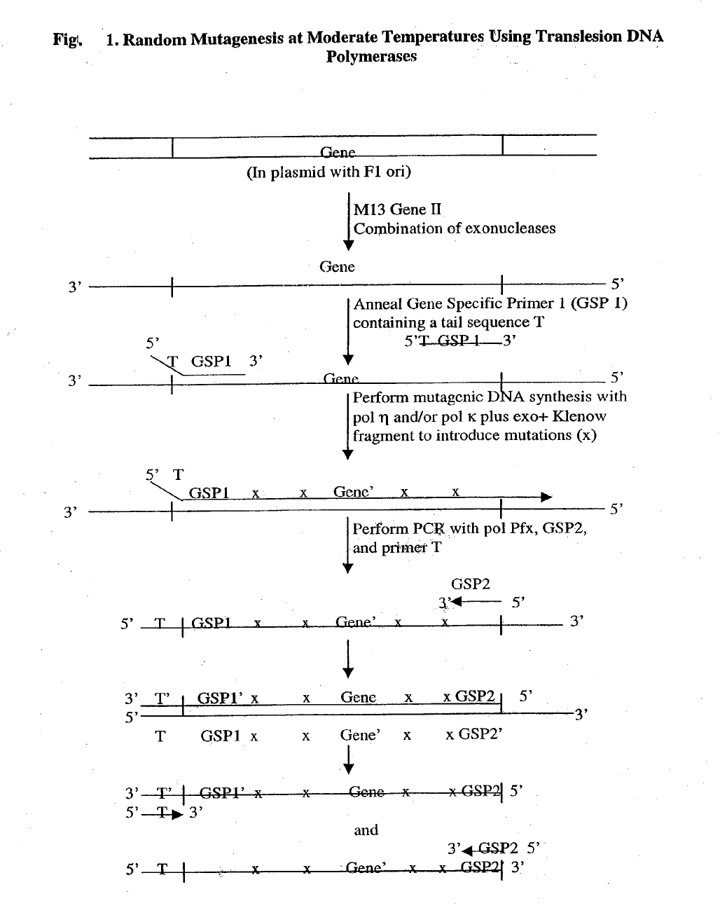

### DETAILED DESCRIPTION OF THE INVENTION

### Definitions

In the description that follows, a number of terms used in recombinant DNA technology are utilized extensively. In order to provide a clearer and consistent understanding of the specification and claims, including the scope to be given such terms, the following definitions are provided.

Translesion DNA Polymerase. As used herein, the term “Translesion DNA Polymerase” refers to members of the UmuC/DinB/Rad30/Rev1 Superfamily of DNA polymerases or refers to DNA polymerases with mutation rates greater than 0.5-1×10⁻⁴ mutations per nucleotide incorporated, more preferably, at least 9×10⁻³, at least 8×10⁻³, at least 7×10⁻³, at least 6×10⁻³, at least 5×10⁻³, at least 4×10⁻³, at least 3×10⁻³, at least 2×10⁻³, at least 1×10⁻³, at least 9×10⁻², at least 8×10⁻², at least 7×10⁻², at least 6×10⁻², at least 5×10⁻², at least 4×10⁻², at least 3×10⁻², at least 2×10⁻², at least 1×10⁻², at least 9×10⁻¹, at least 8×10⁻¹, at least 7×10⁻¹, at least 6×10⁻¹, at least 5×10⁻¹, at least 4×10⁻¹, at least 2×10⁻¹, and at least 1×10⁻¹, and may preferably be in the range of 9×10⁻³ to 1×10⁻¹, 8×10⁻³ to 2×10⁻¹, 7×10⁻³ to 3×10⁻¹, 6×10⁻³ to 4×10⁻¹, 5×10⁻³ to 5×10⁻¹, 4×10⁻³ to 6×10⁻¹, 3×10⁻³ to 7×10⁻¹, 2×10⁻³ to 8×10⁻¹, 1×10⁻³ to 9×10⁻¹, 9×10⁻² to 1×10⁻², 8×10⁻² to 2×10⁻², 7×10⁻² to 3×10⁻², and 6×10⁻² to 4×10⁻², and may preferably be in the range 9×10⁻³ to 8×10⁻³, 9×10⁻³ to 7×10⁻³, 9×10⁻³ to 6×10⁻³, 9×10⁻³ to 5×10⁻³, 9×10⁻³ to 4×10⁻³, 9×10⁻³ to 3×10⁻³, 9×10⁻³ to 2×10⁻³, 9×10⁻³ to 1×10⁻³, 9×10⁻³ to 9×10⁻², 9×10⁻³ to 8×10⁻², 9×10⁻³ to 7×10⁻², 9×10⁻³ to 6×10⁻², 9×10⁻³ to 5×10⁻², 9×10⁻³ to 4×10⁻², 9×10⁻³ to 3×10⁻², 9×10⁻³ to 2×10⁻², 9×10⁻³ to 1×10^{31 2}, 9×10⁻³ to 9×10⁻¹, 9×10⁻³ to 8×10⁻¹, 9×10⁻³ to 7×10⁻¹, 9×10⁻³ to 6×10⁻¹, 9×10⁻³ to 5×10⁻¹, 9×10⁻³ to 4×10⁻¹, 9×10⁻³ to 3×10⁻¹, 9×10⁻³ to 2×10⁻¹, and 9×10⁻³ to 1×10⁻¹, and may preferably be in the range 8×10⁻³ to 7×10⁻³, 8×10⁻³ to 6×10⁻³, 8×10⁻³ to 5×10⁻³, 8×10⁻³ to 4×10⁻³, 8×10⁻³ to 3×10⁻³, 8×10⁻² to 2×10⁻³, 8×10⁻³ to 1×10⁻¹, 8×10⁻³ to 9×10⁻², 8×10⁻³ to 8×10⁻², 8×10⁻³ to 7×10⁻², 8×10⁻³ to 6×10⁻², 8×10⁻³ to 5×10⁻², 8×10⁻³ to 4×10⁻², 8×10⁻³ to 3×10⁻², 8×10⁻³ to 2×10⁻², 8×10⁻³ to 1×10⁻², 8×10⁻³ to 9×10⁻¹, 8×10⁻³ to 8×10⁻¹, 8×10⁻³ to 7×10⁻¹, 8×10⁻³ to 6×10⁻¹, 8×10⁻³ to 5×10⁻¹, 8×10⁻³ to 4×10⁻¹, 8×10⁻³ to 3×10⁻¹, 8×10⁻³ to 2×10⁻¹, and 8×10⁻³ to 1×10⁻¹, and may preferably be in the range 7×10⁻³ to 6×10⁻³, 7×10⁻³ to 5×10⁻³, 7×10⁻³ to 4×10⁻³, 7×10⁻³ to 3×10⁻³, 7×10⁻³ to 2×10⁻³, 7×10⁻³ to 1×10⁻³, 7×10⁻³ to 9×10⁻², 7×10⁻³ to 8×10⁻², 7×10⁻³ to 7×10⁻², 7×⁻³ to 6×10⁻², 7×10^{31 3} to 5×10⁻², 7×10⁻³ to 4×10⁻², 7×10⁻³ to 3×10⁻², 7×10⁻³ to 2×10⁻², 7×10⁻³ to 1×10⁻², 7×10⁻³ to 9×10⁻¹, 7×10⁻³ to 8×10⁻¹, 7×10⁻³ to 7×10⁻¹, 7×10⁻³ to 6×10⁻¹, 7×10⁻³ to 5×10⁻¹, 7×10⁻³ to 4×10⁻¹, 7×10⁻³ to 3×10⁻¹, 7×10⁻³ to 2×10⁻¹, and 7×10⁻³ to 1×10⁻¹, and may preferably be in the range 6×10⁻³ to 5×10⁻³, 6×10⁻³ to 4×10⁻³, 6×10⁻³ to 3×10⁻³, 6×10⁻³ to 2×10⁻³, 6×10⁻³ to 1×10⁻³, 6×10⁻³ to 9×10⁻², 6×10⁻³ to 8×10⁻², 6×10⁻³ to 7×10⁻², 6×10⁻³ to 6×10⁻², 6×10⁻³ to 5×10⁻², 6×10⁻³ to 4×10⁻², 6×10⁻³ to 3×10⁻², 6×10⁻³ to 2×10⁻², 6×10⁻³ to 1×10⁻², 6×10⁻³ to 9×10⁻¹, 6×10⁻³ to 8×10⁻¹, 6×10⁻³ to 7×10⁻¹, 6×10⁻³ to 6×10⁻³, 6×10⁻³ to 5×10⁻¹, 6×10⁻³ to 4×10⁻¹, 6×10⁻³ to 3×10⁻¹, 6×10⁻³ to 2×10⁻¹, and 6×10⁻³ to 1×10⁻¹, and may preferably be in the range 5× to 4×10⁻³, 5× to 3×10⁻³, 5×10⁻³ to 2×10⁻³, 5×10⁻³ to 1×10⁻³, 5×10⁻³ to 9×10⁻², 5×10⁻³ to 8×10⁻², 5×10⁻³ to 7×10⁻², 5×10⁻³ to 6×10⁻², 5×10⁻³ to 5×10⁻², 5×10⁻³ to 4×10⁻², 5×10⁻³ to 3×10⁻², 5× to 2×10⁻², 5×10⁻³ to 1×10⁻², 5× to 9×10⁻¹, 5×10⁻³ to 8×10⁻¹, 5×10⁻³ to 7×10⁻¹, 5×10⁻³ to 6×10⁻¹, 5×10⁻³ to 5×10⁻¹, 5×10⁻³ to 4×10⁻¹, 5× to 3×10⁻¹, 5×10⁻³ to 2×10⁻¹, and 5× to 1×10⁻¹, and may preferably be in the range 4×10⁻³ to 3×10⁻³, 4×10⁻³ to 2×10⁻³, 4×10⁻³ to 1×10⁻³, 4×10⁻³ to 9×10⁻², 4×10⁻³ to 8×10⁻², 4×10⁻³ to 7×10⁻², 4×10⁻³ to 6×10⁻², 4×10⁻³ to 5×10⁻², 4×10⁻³ to 4×10⁻², 4×10⁻³ to 3×10⁻², 4×10⁻³ to 2×10⁻², 4× to 1×10⁻², 4×10⁻³ to 9×10⁻¹, 4×10⁻³ to 8×10⁻¹, 4×10⁻³ to 7×10⁻¹, 4×10⁻³ to 6×10⁻¹, 4×10⁻³ to 5×10⁻¹, 4×10⁻³ to 4×10⁻¹, 4×10⁻³ to 3×10⁻¹, 4×10⁻³ to 2×10⁻¹, and 4× to 1×10−1, and may preferably be in the range 3×10⁻³ to 2×10⁻³, 3× to 1×10⁻³, 3×10⁻³ to 9×10⁻², 3×10−3 to 8×10⁻², 3×10⁻³ to 7×10⁻², 3× to 6×10⁻², 3× to 5×10⁻², 3×10⁻³ to 4×10⁻², 3×10⁻³ to 3×10⁻², 3×10⁻³ to 2×10⁻², 3×10⁻³to 1×10⁻², 3×10⁻³ to 9×10⁻¹, 3×10⁻³ to 8×10⁻¹, 3×10⁻³ to 7×10⁻¹, 3×10⁻³ to 6×10⁻¹, 3×10^{31 3} to 5×10⁻¹, 3×10⁻³ to 4×10⁻¹, 3×10⁻³ to 3×10⁻¹, 3×10⁻³ to 2×10⁻¹, and 3×10⁻³ to 1×10⁻³ and may preferably be in the range 2× to 1×10⁻³, 2×10⁻³ to 9×10⁻², 2× to 8×10⁻², 2×10⁻³ to 7×10⁻², 2×10⁻³ to 6×10⁻², 2×10⁻³ to 5×10⁻², 2×10⁻³ to 4×10⁻², 2×10⁻³ to 3×10⁻², 2×10⁻³ to 2×10⁻², 2×10⁻³ to 1×10⁻², 2×10⁻³ to 9×10⁻¹, 2×10⁻³ to 8×10⁻¹, 2×10⁻³ to 7×10⁻¹, 2×10⁻³ to 6×10⁻¹, 2×10³ to 5×10⁻¹, 2×10⁻³ to 4×10⁻¹, 2×10⁻³ to 3×10⁻¹, 2×10⁻³ to 2×10⁻¹, and 2× to 1×10⁻¹, and may preferably be in the range 1×10⁻³ to 9×10⁻², 1×⁻³ to 8×10⁻², 1×10⁻³ to 7×10⁻², 1×10⁻³ to 6×10⁻², 1×10⁻³ to 5×10⁻², 1×10⁻³ to 4×10⁻², 1×10⁻³ to 3×10⁻², 1×10⁻³ to 2×10⁻², 1×10⁻³ to 1×10⁻², 1×10⁻³ to 9×10⁻¹, 1×10⁻³ to 8×10⁻¹, 1×10⁻³ to 7×10⁻¹, 1×10⁻³ to 6×10⁻¹, 1×10⁻³ to 5×10⁻¹, 1×10⁻³ to 4×10⁻¹, 1×10⁻³ to 3×10⁻¹, 1×10⁻³ to 2×10⁻¹, and 1×10⁻¹ to 1×10⁻¹, and may preferably be in the range 9×10⁻² to 8×10⁻², 9×10⁻² to 7×10⁻², 9×10⁻² to 6×10⁻², 9×10⁻² to 5×10⁻², 9×10⁻² to 4×², 9×10⁻² to 3×10⁻², 9×10⁻² to 2×9×10⁻² to 1×10⁻², 9×10⁻² to 9×10⁻¹, 9×10⁻² to 8×10⁻¹, 9×10⁻² to 7×10⁻¹, 9×10⁻² to 6×10⁻¹, 9×10⁻² to 5×10⁻¹, 9×10⁻² to 4×10⁻¹, 9×10⁻² to 3×10⁻¹, 9×10⁻² to 2×10⁻¹, and 9×10⁻² to 1×10⁻¹, and may preferably be in the range 8×10⁻² to 7×10⁻², 8×10⁻² to 6×10⁻², 8×10⁻² to 5×10⁻², 8×10⁻² to 4×10⁻², 8×10⁻² to 3×10⁻², 8×10⁻² to 2×10⁻², 8×10⁻² to 1×10⁻², 8×10⁻² to 9×10⁻¹, 8×10⁻² to 8×10⁻¹, 8×10⁻² to 7×10⁻¹, 8×10⁻² to 6×10⁻¹, 8×10⁻² to 5×10⁻¹, 8×10⁻² to 4×10⁻¹, 8×10⁻² to 3×10⁻¹, 8×10⁻² to 2×10⁻¹, and 8×10⁻² to 1×10⁻¹, and may preferably be in the range 7×10⁻² to 6×10⁻², 7×10⁻² to 5×10⁻², 7×10⁻² to 4×10⁻², 7×10⁻² to 3×10⁻², 7×10⁻² to 2×10⁻², 7×10⁻² to 1×10⁻², 7×10⁻² to 9×10⁻¹, 7×10⁻³ to 8×10⁻¹, 7×10⁻² to 7×10⁻¹, 7×10⁻² to 6×10⁻¹, 7×10⁻² to 5×10⁻¹, 7×10⁻² to 4×10⁻¹, 7×10⁻² to 3×10⁻¹, 7×10⁻² to 2×10⁻¹, and 7×10⁻² to 1×10⁻¹, and may preferably be in the range 6×10⁻² to 5×10⁻², 6×10⁻² to 4×10⁻², 6×10⁻² to 3×10⁻², 6×10⁻² to 2×10⁻², 6×10⁻² to 1×10⁻², 6×10⁻² to 9×10⁻¹, 6×10⁻² to 8×10⁻¹, 6×10⁻² to 7×10⁻¹, 6×10⁻² to 6×10⁻¹, 6×10⁻² to 5×10⁻¹, 6×10⁻² to 4×10⁻¹, 6×10⁻² to 3×10⁻¹, 6×10⁻² to 2×10⁻¹, and 6×10⁻² to 1×10⁻¹, and may preferably be in the range 5×10⁻² to 4×10⁻², 5×10⁻² to 3×10⁻², 5×10⁻² to 2×10⁻², 5×10⁻² to 1×10⁻², 5×10⁻² to 9×10⁻¹, 5×10⁻² to 8×10⁻¹, 5×10⁻² to 7×10⁻¹, 5×10⁻² to 6×10⁻¹, 5×10⁻² to 5×10⁻¹, 5×10⁻² to 4×10⁻¹, 5×10⁻² to 3×10⁻¹, 5×10⁻² to 2×10⁻¹, and 5×10⁻² to 1×10⁻¹, and may preferably be in the range 4×10⁻² to 3×10⁻², 4×10⁻² to 2×10⁻², 4×10⁻² to 1×10⁻², 4×10⁻² to 9×10⁻¹, 4×10⁻² to 8×10⁻¹, 4×10⁻² to 7×10⁻¹, 4×10⁻² to 6×10⁻¹, 4×10⁻² to 5×10⁻¹, 4×10⁻² to 4×10⁻¹, 4×10⁻² to 3×10⁻¹, 4×10⁻² to 2×10⁻¹, and 4×10⁻² to 1×10⁻¹, and may preferably be in the range 3×10⁻² to 2×10⁻², 3×10⁻² to 1×10⁻², 3×10⁻² to 9×10⁻¹, 3×10⁻² to 8×10⁻¹, 3×10⁻² to 7×10⁻¹, 3×10⁻² to 6×10⁻¹, 3×10⁻² to 5×10⁻¹, 3×10⁻² to 4×10⁻¹, 3×10⁻² to 3×10⁻¹, 3×10⁻² to 2×10⁻¹, and 3×10⁻² to 1×10⁻¹, and may preferably be in the range 2×10⁻² to 1×10⁻², 2×10⁻² to 9×10⁻¹, 2×10⁻² to 8×10⁻¹, 2×10⁻² to 7×10⁻¹, 2×10⁻² to 6×10⁻¹, 2×10⁻² to 5×10⁻¹, 2×10⁻² to 4×10⁻¹, 2×10⁻² to 3×10⁻¹, 2×10⁻² to 2×10⁻¹, and 2×10⁻² to 1×10⁻¹, and may preferably be in the range 1×10⁻² to 9×10⁻¹, 1×10⁻² to 8×10⁻¹, 1×10⁻² to 7×10⁻¹, 1×10⁻² to 6×10⁻¹, 1×10⁻² to 5×10⁻¹, 1×10⁻² to 4×10⁻¹, 1×10⁻² to 3×10⁻¹, 1×10⁻² to 2×10⁻¹, and 1×10⁻² to 1×10⁻¹, and may preferably be in the range 9×10⁻¹ to 8×10⁻¹, 9×10⁻¹ to 7×10⁻¹, 9×10⁻¹ to 6×10⁻¹, 9×10⁻¹ to 5×10⁻¹, 9×10⁻¹ to 4×10⁻¹, 9×10⁻¹ to 3×10⁻¹, 9×10⁻¹ to 2×10⁻¹, and 9×10⁻¹ to 1×10⁻¹, and may preferably be in the range 8×10⁻¹ to 7×10⁻¹, 8×10⁻¹ to 6×10⁻¹, 8×10⁻¹ to 5×10⁻¹, 8×10⁻¹ to 4×10⁻¹, 8×10⁻¹ to 3×10⁻¹, 8×10⁻¹ to 2×10⁻¹, and 8×10⁻¹ to 1×10⁻¹, and may preferably be in the range 7×10⁻¹ to 6×10⁻¹, 7×10⁻¹ to 5×10⁻¹, 7×10⁻¹ to 4×7×10⁻¹ to 3×10⁻¹, 7×10⁻¹ to 2×10⁻¹, and 7×10⁻¹ to 1×10⁻¹, and may preferably be in the range 6×10⁻¹ to 5×10⁻¹, 6×10⁻¹ to 4×10⁻¹, 6×10⁻¹ to 3×10⁻¹, 6×10⁻¹ to 2×10⁻¹, and 6×10⁻¹ to 1×10⁻¹, and may preferably be in the range 5×10⁻¹ to 4×10⁻¹, 5×10⁻¹ to 3×10⁻¹, 5×10⁻³ to 2×10⁻¹, and 5×10⁻¹ to 1×10⁻¹, and may preferably be in the range 4×10⁻¹ to 3×10⁻¹, 4×10⁻¹ to 2×10⁻¹, and 4×10⁻¹ to 1×10⁻¹, and may preferably be in the range 3×10⁻¹ to 2×10⁻¹, and 3×10⁻¹ to 1×10⁻¹, and may preferably be in the range 2×10⁻¹ to 1×10⁻¹.

Non-Translesion DNA Polymerase. As used herein, the term “non-translesion DNA polymerase” refers to any polymerase other than a Translesion polymerase. Non-Translesion polymerases include polymerases from the A superfamily, B superfamily, C superfamily, and X superfamily. Non-Translesion polymerase also includes any reverse transcriptases (RT) which may be reduced or substantially reduced in RNaseH activity or may lack RnaseH activity. Non-Translesion polymerases include E. coli pol I, pol T7, pol T5, pol Taq, pol Tth, pol Tne, reverse transcriptases (particularly retroviral reverse transcriptases (such as M-MLVRT, AMV-RT, RSU-RT and the like)), and eukaryotic pol γ (gamma), E. coli pol II, eukaryotic pol α (alpha), eukaryotic δ (delta), eukaryotic ε (epsilon), pol T4, pol Φ29, pol Pfu, and pol KOD (Pfx), E. coli pol III α subunit, eukaryotic pol β (beta), eukaryotic λ (lambda), eukaryotic μ (mu), and TdT.

Library. As used herein, the term “library” or “nucleic acid library” means a set of nucleic acid molecules (circular or linear) representative of all or a portion of the DNA content of an organism (a “genomic library”), or a set of nucleic acid molecules representative of all or a portion of the expressed genes (a “cDNA library”) in a cell, tissue, organ or organism. Such libraries may or may not be contained in one or more vectors.

Vector. As used herein, a “vector” is a plasmid, cosmid, phagemid or phage DNA or other DNA molecule which is able to replicate autonomously in a host cell, and which is characterized by one or a small number of restriction endonuclease recognition sites at which such DNA sequences may be cut in a determinable fashion without loss of an essential biological function of the vector, and into which DNA may be inserted in order to bring about its replication and cloning. The vector may further contain a marker suitable for use in the identification of cells transformed with the vector. Markers, for example, include but are not limited to tetracycline resistance or ampicillin resistance.

Primer. As used herein, “primer” refers to a nucleic acid molecule that is extended by covalent bonding of nucleotide monomers during amplification or polymerization of a DNA molecule. A primer may be attached to the DNA molecule to be amplified, via hairpin or other means, or it may be a separate molecule.

Template. The term “template” as used herein refers to a double-stranded or single-stranded nucleic acid (RNA or DNA) molecule which is to be amplified (copied), synthesized, mutagenized, or modified. In the case of a double-stranded molecule, denaturation of its strands to form a first and a second strand is performed before these molecules may be amplified, copied, mutagenized, or modified. A primer, complementary to a portion of a template is hybridized under appropriate conditions and a DNA polymerase may then synthesize a nucleic acid molecule complementary to said template or a portion thereof. The newly synthesized molecule, according to the invention, may be equal to or shorter in length than the original template. Mismatch incorporation and/or insertions and/or deletions during the synthesis or extension of the newly synthesized molecule may result in one or a number of changes or mismatched base pairs. Thus, the synthesized molecule need not be exactly complementary to the template. The template may be one or more molecules, such as a polulation of molecules.

Incorporating. The term “incorporating” as used herein means becoming a part of a nucleic acid molecule such as a nucleotide becoming part of a DNA primer or probe or other DNA molecule. In a preferred embodiment, one or more modified nucleotides are incorporated into a DNA molecule such as a probe or primer. In another preferred embodiment, one or more modified nucleotides are incorporated into a DNA molecule for use in DNA array technology.

Random Mutagensis. The term “random mutagenesis” refers to non-directed mismatch incorporation that may occur anywhere on a nucleic acid molecule. A mismatch may be any type of misincorporation such as a transition, a transversion, a deletion, or an insertion. Mismatches are also referred to herein as mutations. A nucleic acid produced by random mutagenesis may be referred to herein as “randomized” or “mutagenized” or grammatical equivalents thereof.

Amplification. As used herein “amplification” refers to any in vitro method for increasing the number of copies of a nucleotide sequence with the use of a polymerase. Amplification may be linear or may be exponential. Nucleic acid amplification results in the incorporation of nucleotides into a DNA molecule such as a primer or probe thereby forming a new molecule complementary to a template. The formed nucleic acid molecule and its template may be used as templates to synthesize additional nucleic acid molecules. As used herein, one amplification reaction may consist of many rounds of replication. DNA amplification reactions include, for example, polymerase chain reactions (PCR). One PCR reaction may consist of 5 to 100 “cycles” of denaturation and synthesis of a DNA molecule.

Oligonucleotide. “Oligonucleotide” refers to a synthetic or natural molecule comprising a covalently linked sequence of nucleotides which are joined by a phosphodiester bond between the 3′ position of the deoxyribose or ribose of one nucleotide and the 5′ position of the deoxyribose or ribose of the adjacent nucleotide.

Nucleotide. As used herein “nucleotide” refers to a base-sugar-phosphate combination. Nucleotides are monomeric units of a nucleic acid sequence (DNA and RNA). The term nucleotide includes ribonucleoside triphosphates (NTPs) ATP, UTP, CTG, GTP and deoxyribonucleoside triphosphates (dNTPs) such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddATP, ddITP, and ddTTP. According to the present invention, a “nucleotide” may be unlabeled or detectably labeled by well known techniques. Detectable labels include, for example, radioactive labels, metal labels such as gold, magnetic resonance labels, dye labels, fluorescent labels, chemiluminescent labels, electrochemiluminescent labels (ECL; see U.S. Pat. Nos. 6,174,709 and 5,610,017), bioluminescent labels, enzyme labels, antigenic determinants detectable by an antibody, biotin labels, and digoxigenin labels (DIG). Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue™, Oreg. Green™, Texas Red™, Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TA MRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink™ DeoxyNucleotides, FluoroLink Cy3-dCTP, FluOroLink Cy5-dCTP, FluoroLink Fluor X-dCTP, FluoroLink Cy3-dUTP, and FluoroLink Cy5-dUTP available from Amersham Arlington Heights, Ill.; Fluorescein-15-dATP, Fluorescein-12-dUTP, Tetramethyl-rodamine-6-dUTP, IR₇₇₀-9-dATP, Fluorescein-12-ddUTP, Fluorescein-12-UTP, and Fluorescein-15-2′-dATP available from Boehringer Mannheim Indianapolis, Ind.; and ChromaTide™ Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oreg. Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. DIG labels include digoxigenin-11-UTP available from Boehringer Mannheim, Indianapolis, Ind., and biotin labels include biotin-21-UTP and amino-7-dUTP available from Clontech, Palo Alto, Calif. The term nucleotide includes modified nucleotides.

Modified Nucleotide. The term “modified nucleotide” refers to a nucleotide other than dATP, dCTP, dUTP, dGTP, and dTTP. Thus, the term modified nucleotide excludes dATP, dCTP, dUTP, dGTP, and dTTP. The term modified nucleotide includes ddNTPs, and nucleotide derivatives such as ddNTP derivatives, dNTP derivatives, and NTP derivatives. Modified nucleotides also include labeled nucleotides. Preferred modified nucleotides include nucleotides that are bulky relative to dATP, dCTP, dUTP, dGTP, and dTTP. Many examples of modified nucleotides are disclosed in U.S. Pat. No. 6,200,757.

Hybridization. The terms “hybridization” and “hybridizing” refers to base pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used.

Unit. The term “unit” as used herein refers to the activity of an enzyme. When referring, for example, to a thermostable DNA polymerase, one unit of activity is the amount of enzyme that will incorporate 10 nanomoles of dNTPs into acid-insoluble material (i.e., DNA or RNA) in 30 minutes under standard primed DNA synthesis conditions.

Probes. The term “probes” refers to single or double stranded nucleic acid molecules or oligonucleotides which are used to detect or analyze a nucleic acid of interest. In some embodiments, a probe is unlabeled. For example, in array technology, nucleic acid probes bound to the substrate (e.g., chip) are unlabeled and the nucleic acid of interest is labeled. In other embodiments, a probe is detectably labeled by one or more detectable markers or labels. For example, in Southern and northern blot analysis, the probe is labeled and the nucleic acid of interest is unlabeled. Such labels or markers may be the same or different and may include radioactive labels, magnetic resonance labels, dye labels, fluorescent labels, chemiluminescent labels, electrochemiluminescent labels (ECL), bioluminescent labels, enzyme labels, antigenic determinants detectable by an antibody, biotin labels, and digoxigenin labels (DIG), although one or more fluorescent labels (which are the same or different) are preferred in accordance with the invention. Probes have specific utility in the detection of nucleic acid molecules by hybridization and thus may be used in diagnostic assays. Electrochemiluminescent (ECL) labels are those which become luminescent species when acted on electrochemically. They provide a sensitive and precise measurement of the presence and concentration of an analyte of interest. In such techniques, the sample is exposed to a voltammetric working electrode in order to trigger luminescence. The light produced by the label is measured and indicates the presence or quantity of the analyte. Such ECL techniques are described in U.S. Pat. No. 5,610,017, WO86/02734 and WO87/06706.

Expression. Expression is the process by which a polynucleotide produces a mRNA or a polypeptide. It involves transcription of the polynucleotide into messenger RNA (mRNA) and, in the case of polypeptide expression, translation of such mRNA into polypeptide(s).

Recombinant host. The term “recombinant host” as used herein refers to any prokaryotic or eukaryotic microorganism which contains the desired cloned genes in an expression vector, cloning vector or any other nucleic acid molecule. The term “recombinant host” is also meant to include those host cells which have been genetically engineered to contain the desired gene on a host chromosome or in the host genome.

Host. The term “host” as used herein refers to any prokaryotic or eukaryotic microorganism that is the recipient of a replicable expression vector, cloning vector or any nucleic acid molecule including the inhibitory nucleic acid molecules of the invention. The nucleic acid molecule may contain, but is not limited to, a structural gene, a promoter and/or an origin of replication.

Promoter. The term “promoter” as used herein refers to a DNA sequence generally described as the 5′ region of a gene, located proximal to start the codon. At the promoter region, transcription of an adjacent gene(s) is initiated.

Gene. The term “gene” as used herein refers to a DNA sequence that contains information necessary for expression of a polypeptide or protein. It includes the promoter and the structural gene as well as other sequences involved in expression of the protein.

Structural gene. The term “structural gene” as used herein refers to a DNA sequence that is transcribed into messenger RNA that is then translated into a sequence of amino acids characteristic of a specific polypeptide.

Operably linked. The term “operably linked” as used herein means that the promoter is positioned to control the initiation of expression of the polypeptide encoded by the structural gene.

Substantially Pure. As used herein “substantially pure” means that the desired purified molecule such as a protein or nucleic acid molecule (including the inhibitory nucleic acid molecule of the invention) is essentially free from contaminants which are typically associated with the desired molecule. Contaminating components may include, but are not limited to, compounds or molecules which may interfere with the inhibitory or synthesis reactions of the invention, and/or that degrade or digest the inhibitory nucleic acid molecules of the invention (such as nucleases including exonucleases and endonucleases) or that degrade or digest the synthesized or amplified nucleic acid molecules produced by the methods of the invention.

Thermostable. As used herein “thermostable” refers to a DNA polymerase which is more resistant to inactivation by heat. DNA polymerases synthesize the formation of a DNA molecule complementary to a single-stranded DNA template by extending a primer in the 5′-3′-direction. This activity for mesophilic DNA polymerases may be inactivated by heat treatment. For example, T5 DNA polymerase activity is totally inactivated by exposing the enzyme to a temperature of 90° C. for 30 seconds. As used herein, a thermostable DNA polymerase activity is more resistant to heat inactivation than a mesophilic DNA polymerase. However, a thermostable DNA polymerase does not mean to refer to an enzyme which is totally resistant to heat inactivation and thus heat treatment may reduce the DNA polymerase activity to some extent. A thermostable DNA polymerase typically will also have a higher optimum temperature than mesophilic DNA polymerases.

3′-to-5′ Exonuclease Activity. “3′-to-5′ exonuclease activity” is an enzymatic activity well known to the art. This activity is often associated with DNA polymerases and is thought to be involved in a DNA replication “editing” or correction mechanism.

A “DNA polymerase substantially reduced in 3′-to-5′ exonuclease activity” is defined herein as either (1) a mutated DNA polymerase that has about or less than 10%, or preferably about or less than 1%, of the 3′-to-5′ exonuclease activity of the corresponding unmutated, wild-type enzyme, or (2) a DNA polymerase having a 3′-to-5′ exonuclease specific activity which is less than about 1 unit/mg protein, or preferably about or less than 0.1 units/mg protein. A unit of activity of 3′-to-5′ exonuclease is defined as the amount of activity that solubilizes 10 nmoles of substrate ends in 60 min. at 37° C., assayed as described in the “BRL 1989 Catalogue & Reference Guide”, page 5, with HhaI fragments of lambda DNA 3′-end labeled with [³H]dTTP by terminal deoxynucleotidyl transferase (TdT). Protein is measured by the method of Brandford, Anal. Biochem. 72:248 (1976). As a means of comparison, natural, wild-type T5-DNA polymerase (DNAP) or T5-DNAP encoded by pTTQ19-T5-2 has a specific activity of about 10 units/mg protein while the DNA polymerase encoded by pTTQ19-T5-2(Exo−) (U.S. Pat. No. 5,270,179) has a specific activity of about 0.0001 units/mg protein, or 0.001% of the specific activity of the unmodified enzyme, a 10⁵-fold reduction. Polymerases used in accordance with the invention may lack or may be substantially reduced in 3′ exonuclease activity.

5′-to-3′ Exonuclease Activity. “5′-to-3′ exonuclease activity” is also enzymatic activity well known in the art. This activity is often associated with DNA polymerases, such as E. coli Poll and Tag DNA polymerase.

A “polymerase substantially reduced in 5′-to-3′ exonuclease activity” is defined herein as either (1) mutated or modified polymerase that has about or less than 10%, or preferably about or less than 1%, of the 5′-to-3′ exonuclease activity of the corresponding unmutated, wild-type enzyme, or (2) a polymerase having 5′-to-3′ exonuclease specific activity which is less than about 1 unit/mg protein, or preferably about or less than 0.1 units/mg protein.

Both of the 3′-to-5′ and 5′-to-3′ exonuclease activities can be observed on sequencing gels. Active 5′-to-3′ exonuclease activity will produce different size products in a sequencing gel by removing mono-nucleotides and longer products from the 5′-end of the growing primers. 3′-to-5′ exonuclease activity can be measured by following the degradation of radiolabeled primers in a sequencing gel. Thus, the relative amounts of these activities (e.g., by comparing wild-type and mutant or modified polymerases) can be determined with no more than routine experimentation.

Distributive. As used herein, “distributive” polymerases generally incorporate one nucleotide before disassociating from the template nucleic acid molecule.

Non processive. As used herein, “non processive” polymerases generally incorporate fewer than ten (10) nucleotides before disassociating from the template nucleic acid molecule.

Processive. As used herein, “processive” polymerases generally incorporate hundreds of nucleotides before disassociating from the template nucleic acid molecule. “Moderately processive” polymerases generally incorporate ten (10) or more nucleotides but fewer than hundreds of nucleotides before disassociating from the template nucleic acid molecule.

Other terms used in the fields of recombinant DNA technology and molecular and cell biology as used herein will be generally understood by one of ordinary skill in the applicable arts.

### Overview

The present invention provides kits, compositions and methods useful in overcoming limitations in random mutagenesis and incorporation of modified nucleotides. The present invention achieves previously unattainable mutation frequencies of 2 to 20 base pairs per 1,000 nucleotides in one round of mutagenesis. The invention also facilitates the production of modified, e.g., labeled, nucleic acid molecules not heretofore possible.

Mutagenesis. The methods of the present invention relate generally to methods of synthesizing and/or amplifying nucleic acid molecules. In one aspect, the invention relates to kits and methods for incorporating mutations, preferably randomly, in DNA molecules. In this aspect, a template nucleic acid molecule and a Translesion DNA polymerase are incubated under conditions sufficient to allow synthesis of a complementary nucleic acid molecule. Such conditions generally require at least one primer and dNTPs, and may also require salts and/or accessory proteins. A Translesion DNA polymerase incorporates at least one random mutation in the complementary nucleic acid molecule. One or more rounds of synthesis may be performed to incorporate random mutations. The mutation rate may be altered up or down by including Translesion DNA polymerases and non-translesion DNA polymerases with various misincorporation rates in the method. The resulting complementary nucleic acid molecules or population of nucleic acid molecules (mutagenized nucleic acid molecules) may be further amplified using standard amplification techniques such as PCR.

The invention further provides mutagenized nucleic acids produced by the methods of the invention. Such mutagenized nucleic acid molecules may be single or double stranded. Mutagenized nucleic acids are useful for structure-function studies and for optimizing encoded mRNA and polypeptides. Such molecules, especially polypeptides, can be assayed for improved enzymatic activities, receptor properties, ligand interactions, antibiotic or antiviral properties, vaccine efficacy, or antibody binding affinity. The invention also provides polypeptides encoded by the mutagenized nucleic acids of the invention.

Modified Polynucleotides. In another aspect, the present invention relates to kits and methods of synthesizing modified nucleic acid molecules. In this aspect, a template nucleic acid molecule, a Translesion DNA polymerase, and a modified nucleotide are incubated under conditions sufficient to allow synthesis of a complementary nucleic acid molecule. Such conditions generally require at least one primer and dNTPs, and may also require salts and/or accessory proteins. The Translesion DNA polymerase incorporates the modified nucleotide in the complementary nucleic acid molecule. One or more rounds of synthesis may be used.

In accordance with the invention, the amount of modified, e.g., labeled, product is preferably measured based on percent incorporation of the modification of interest into synthesized product as may be determined by one skilled in the art, although other means of measuring the amount or efficiency of modification will be recognized by one of ordinary skill in the art. The invention provides for enhanced or increased percent incorporation of modified nucleotide during synthesis of a nucleic acid molecule from a template

The invention also provides modified nucleic acid molecules produced according to the above-described methods. Such modified nucleic acid molecules may be single or double stranded. Modified nucleic acid molecules include labeled nucleic acid molecules and are useful as detection probes. Depending on the modified nucleotide(s) used during synthesis, the modified molecules may contain one or a number of modifications. Where multiple modifications are used, the molecules may comprise a number of the same or different modifications such as labels. Thus, one type or multiple different modified nucleotides may be used during synthesis of nucleic acid molecules to provide for the modified nucleic acid molecules of the invention. Such modified nucleic acid molecules will thus comprise one or more modified nucleotides (which may be the same or different).

### DNA Polymerases

A variety of Translesion DNA polymerases may be used in the present methods. Such polymerases include, but are not limited to, vertebrate Translesion DNA polymerases, mammalian Translesion DNA polymerases, animal Translesion DNA polymerases, human Translesion DNA polymerases, mouse Translesion DNA polymerases, C. elegans Translesion DNA polymerases, insect Translesion DNA polymerases, Drosophila Translesion DNA polymerases, bacterial Translesion DNA polymerases, E. coli Translesion DNA polymerases, S. cerevisiae Translesion DNA polymerases, S. pombe Translesion DNA polymerases, eubacterial Translesion DNA polymerases, archaebacterial Translesion DNA polymerases, Thermus thermophilus Translesion DNA polymerases, Thermus aquaticus Translesion DNA polymerases, Thermotoga neopolitana Translesion DNA polymerases, Thermotoga maritima Translesion DNA polymerases, Thermococcus litoralis Translesion DNA polymerases, Pyrococcus furiosus Translesion DNA polymerases, Pyrococcus woosii Translesion DNA polymerases, Pyrococcus sp Translesion DNA polymerases, Bacillus sterothermophilus Translesion DNA polymerases, Bacillus caldophilus Translesion DNA polymerases, Sulfolobus acidocaldarius Translesion DNA polymerases, Thermoplasma acidophilum Translesion DNA polymerases, Thermus flavus Translesion DNA polymerases, Thermus rubor Translesion DNA polymerases, Thermus brockianus Translesion DNA polymerases, Methanobacterium thermoautotrophicum Translesion DNA polymerases, mycobacterium Translesion DNA polymerases, and mutants, variants and derivatives thereof.

Translesion DNA polymerases that may be used in the present methods include any member of the UmuC/DinB/Rad30/Rev1 Superfamily, including Pol IV, Pol V, Pol κ, Pol ζ, Pol η, and Pol ι. The Translesion DNA polymerases used in the present methods may be mesophilic or thermophilic/thermostable. Preferred mesophilic Translesion DNA polymerases include Pol IV and Pol V from E. coli and other bacteria; Pol κ from S. cerevisiae, S. pombe, human, mouse, Drosophila, and the like; Pol ζ from S. cerevisiae, human, mouse, and the like; Pol η from S. cerevisiae, human, mouse, and the like; Pol ι from mouse, human, and the like. Preferred thermophilic Translesion DNA polymerases include Pol IV from B. stearothermophilus, S. sofataricus, and the like.

Preferred Translesion DNA polymerases for use in the random mutagenesis methods of the invention include those with high misincorporation rates such as Pol κ and Pol η, although Translesion DNA polymerases such as Pol V with moderate or relatively low misincorporation rates may also be used. More than one Translesion DNA polymerase may be used in the present methods. For example, two, three, four, five, six, or more Translesion DNA polymerases may be used. Preferred combinations of Translesion DNA polymerases for use in the random mutagenesis methods include Pol ζ with one or more other Translesion DNA polymerases such as Pol κ or Pol η. Thus, for example, Pol ζ may be used in combination with either Pol κ or Pol η or it may be used with both Pol κ and Pol η. Translesion DNA polymerases may also be used in combination with one or more non-translesion DNA polymerases in the present methods, as described below.

Preferred Translesion DNA polymerases for use in synthesizing modified nucleic acid molecules include those able to incorporate nucleotides across from bulky lesions in damaged DNA or those which are able to violate Watson-Crick base pairing, such as Pol ι and Pol η. As noted above, more than one Translesion DNA polymerase may be used in the present methods. For example, two, three, four, five, six, or more Translesion DNA polymerases may be used. Preferred combinations of Translesion DNA polymerases for use in synthesizing modified nucleic acid molecules include Pol ζ with one or more other Translesion DNA polymerases such as Pol ι or Pol η. Translesion DNA polymerases may also be used in combination with non-translesion DNA polymerases in the present methods, as described below.

The ratio of one to another Translesion DNA polymerase may be from 10:1 to 1:10, more specifically, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and 1:10. In methods using more than two Translesion DNA polymerases, the ratios may be from 10:1:1 to 1:10:1 to 1:1:10, and any ratio in between.

Translesion DNA polymerases used in the present invention may be isolated from natural or recombinant sources, by techniques that are well-known in the art (see below), from a variety of cells, cells lines, and bacteria that are available commercially (for example, from American Type Culture Collection, Manassass, Va., and see below) or may be obtained by recombinant DNA techniques using publicly available sequences or degenerate sequences (see below). Random mutagenesis and modified nucleic acid synthesis methods of the invention are carried out under well known conditions for in vitro DNA polymerization, such as those disclosed in the publications below. Random mutagenesis on particular templates may be optimized using in vitro fidelity assays disclosed in the publications below or otherwise known in the art.

The E. coli Pol V (UmuD′₂C) UmuC sequences are disclosed in Kitagawa, Y., et al., Proc. Natl. Acad. Sci. U.S.A. 82(13):4336-4340 (1985); Perry, K. L., et al., Proc. Natl. Acad. Sci. U.S.A. 82(13):4331-4335 (1985); Blattner, F. R., et al., Science 277 (5331):1453-1474 (1997); and GenBank accession no. PO4152. The E. coli. UmuD sequences are disclosed in Kitagawa, Y., et al., Proc. Natl. Acad. Sci. U.S.A. 82(13):5336-4340 (1985); Perry, K. L., et al., Proc. Natl. Acad. Sci. U.S.A. 82(13):4331-4335 (1985); Blattner, F. R., et al., Science 277 (5331):1453-1474 (1997); and GenBank accession no. PO4153. Overexpression and purification of UmuC, UmuD′, and complexes of the two proteins are disclosed in Bruck, 1., et al., J. Biol. Chem. 271:10767-10774 (1996); Tang, M., et al., Proc. Natl. Acad. Sci. USA 95:9755-9760 (1998); Tang, M. et al., Proc. Natl. Acad. Sci. USA 96:8919-8924 (1999); Reuven, N. B., et al., J. Biol. Chem. 274:31763-31766 (1999); Reuven et al. Mol. Cell. 2:191-199 (1998). Conditions for in vitro polymerization using Pol V are disclosed in Tang, M., et al., Proc. Natl. Acad. Sci. USA 95:9755-9760 (1998). In vitro replication fidelity assays using Pol V are disclosed in Maor-Shoshani, A. et al., Proc. Natl. Acad. Sci. USA 97:565-570 (2000); Tang, M., et al., Nature 404:1014-1018 (2000). For the Pol V mutasome, see also RecA*, β,γ-complex, and SSB sources/purification, below. Additionally, ATPγ-S can be substituted for β,γ complex (Pham, P., et al., Nature 409:366-370 (2001)).

The E. coli Pol IV (DinB1; sometimes referred to as DinP) sequences are disclosed in Ohmori, H., et al., Mutat. Res. 347 (1):1-7 (1995) and GenBank accession nos. Q47155 and D38582. Purification of Pol IV is disclosed in Tang, M., et al., Nature 404:1014-1018 (2000); Wagner, J., et al., Mol. Cell. 4: 281-286 (1999). Conditions for in vitro polymeriation using Pol IV are disclosed in Tang, M., et al., Nature 404:1014-1018 (2000); Wagner, J., et al., Mol. Cell. 4: 281-286 (1999). In vitro replication fidelity assays using Pol IV are disclosed in Tang, M., et al., Nature 404:1014-1018 (2000); Wagner, J., et al., Mol. Cell. 4: 281-286 (1999). See also β,γ-complex, and SSB sources/purification, below.

The Sulfolobus sofataricus Pol IV sequences are disclosed in She, Q., et al., Proc. Natl. Acad. Sci. U.S.A. 98 (14):7835-7840 (2001); Kulaeva, 0.1., et al., Mutat. Res. 357:245-253 (1996); and GenBank accession nos. AAK42588 and AE006843.

The S. cerevisiae Pol κ (DinB1; cloned as TRF4) sequences are disclosed in Sadoff, B. U., et al., Genetics 141 (2):465-479 (1995); Vandenbol, M., et al., Yeast 11 (11):1069-1075 (1995). Expression/purification of scPol κ are disclosed in Wang, Z., et al., Science 289:774-779 (2000).

The S. pombe Pol κ sequences are disclosed in GenBank accession nos. CAA19259 and AL023704.

The C. elegans Pol κ sequences are disclosed in Wilson, R., et al., Nature 368:32-38 (1994) and GenBank accession no. P34409.

The mouse Pol κ (DinB1) sequences are disclosed in Gerlach, V. L., et al., Proc. Natl. Acad. Sci. USA 96:11922-11927 (1999); GenBank accession no. AF163571; and Ogi, T., et al., Genes Cells 4:607-618 (1999). Expression/purification of mouse Pol lc are disclosed in Tang, M., et al., Nature 404:1014-1018 (2000); Wagner, J., et al., Mol. Cell. 4: 281-286 (1999); Ohashi, E., et al., Gen. Dev. 14:1589-1594 (2000). Conditions for in vitro polymerization using mouse Pol lc are disclosed in Tang, M., et al., Nature 404:1014-1018 (2000); Wagner, J., et al., Mol. Cell. 4: 281-286 (1999). In vitro replication fidelity assays using mouse Pol κ are disclosed in Tang, M., et al., Nature 404:1014-1018 (2000); Wagner, J., et al., Mol. Cell. 4: 281-286 (1999).

The human Pol κ (also referred to as Pol θ) (DINB1) sequences are disclosed in Gerlach, V. L., et al., Proc. Natl. Acad. Sci. USA 96:11922-11927 (1999); Johnson, R. E., Proc. Natl. Acad. Sci. USA 97:3838-3843 (2000); and GenBank accession no. AF163570. Expression/purification of human Pol κ are disclosed in Tang, M., et al., Nature 404:1014-1018 (2000); Wagner, J., et al., Mol. Cell. 4: 281-286 (1999); Ohashi, E., et al., Gen. Dev. 14:1589-1594 (2000); Ohashi, E., et al., J. Biol. Chem. 275:39678-39684 (2000); Zhang, Y., et al., Nuc. Acids Res. 28:4138-4146 (2000); Gerlach, V. L., et al., J. Biol. Chem. 276:92-98 (2001); Zhang, Y., et al., Nuc. Acids Res. 28:4147-4156 (2000); Johnson, R. E., Proc. Natl. Acad. Sci. USA 97:3838-3843 (2000)). Conditions for in vitro polymerization using human Pol κ are disclosed in Tang, M., et al., Nature 404:1014-1018 (2000); Wagner, J., et al., Mol. Cell. 4: 281-286 (1999); Ohashi, E., et al., Gen. Dev. 14:1589-1594 (2000); Ohashi, E., et al., J. Biol. Chem. 275:39678-39684 (2000); Gerlach, V. L., et al., J. Biol. Chem. 276:92-98 (2001); Zhang, Y., et al., Nuc. Acids Res. 28:4147-4156 (2000); Johnson, R. E., Proc. Natl. Acad. Sci. USA 97:3838-3843 (2000). In vitro replication fidelity assays using human Pol κ are disclosed in Tang, M., et al., Nature 404:1014-1018 (2000); Wagner, J., et al., Mol. Cell. 4: 281-286 (1999); Ohashi, E., et al., Gen. Dev. 14:1589-1594 (2000); Ohashi, E., et al., J. Biol. Chem. 275:39678-39684 (2000); Zhang, Y., et al., Nuc. Acids Res. 28:4147-4156 (2000); Johnson, R. E., Proc. Natl. Acad. Sci. USA 97:3838-3843 (2000). A truncated form of human Pol κ having polymerase activity is disclosed in Ohashi, E., et al., Gen. Dev. 14:1589-1594 (2000); Ohashi, E., et al., J. Biol. Chem. 275:39678-39684 (2000).

S. cerevisiae Pol ζ (Rev 1p, Rev3p, Rev7p): The sequences of scREV1 are disclosed in Larimer, F., et al., J. Bacteriol. 171:230-237 (1989); Goffeau, A., et al., Science 274:546 (1996); Dujon, B., et al., Nature 387:98-102 (1997); and GenBank accession nos. NP_{—}014991 and S67255. Overexpression/purification of Rev1p are disclosed in Nelson J. R., et al., Nature 382:729-731 (1996). The sequences of scREV3 are disclosed in Morrison, A., et al., J. Bacterial. 171:5659 (1989); and GenBank accession no. P14284. Overexpression/purification of scRev3p are disclosed in Nelson, J. R., et al., Science 272:1646-1649 (1996). The sequences of scREV7 are disclosed in Torpey, L. E., et al., Yeast 10:1503 (1994) and Goffeau, A., et al., Science 274:546 (1996). Overexpression and/or purification of scRev7p are disclosed in Nelson, J. R., et al., Science 272:1646-1649 (1996) and GenBank accession nos. NP_{—}012127 and P38927.

Mouse Pol ζ (Rev1, Rev31, Rev7): The mREV1 sequences are disclosed in GenBank accession nos. NP_{—}062516 and AF179302. The mREV3 sequences (originally cloned as Sez4) are disclosed in Kajiwara, K. et al., Biochem. Biophys. Res. Com. 219:795-799 (1996); Van Sloun, P. P. P. H., et al., Mutat. Res. 433:109-116 (1999); and GenBank accession nos. BAA90768 and BAA11461.

Human Pol ζ (REV1, REV3, REV7): The hREV1 sequences are disclosed in Gibbs, P. E. M., Proc. Natl. Acad. Sci. USA 97:4186-4191 (2000); Lin, W., et al., Nucleic Acids Res. 27:4468-4475 (1999), and GenBank no. AF206019. hREV3 sequences are disclosed in Gibbs, P. E. M., et al., Proc. Natl. Acad. Sci. USA 95:6876-6880 (1998); Murakumo, Y., et al., J. Biol. Chem. 275:4391-4397 (2000), and GenBank Nos. AF058701 and AF035537. hREV7 sequences are disclosed in Murakumo, Y., et al., J. Biol. Chem. 275:4391-4397 (2000); and GenBank no. AF157482. hREV7 expression/purification are disclosed in Murakumo, Y., et al., J. Biol. Chem. 275:4391-4397 (2000).

The S. cerevisiae Pol η (Rad30) sequences are disclosed in Goffeau, A., et al., Science 274:546 (1996); Jacq, C., et al., Nature 387(6632 Suppl.):75-78 (1997); and GenBank accession no. NP_{—}010707. Expression/purification of S. cerevisiae Pol η are disclosed in Johnson, R. E., et al., Science 283:1001-1004 (1999); Johnson, R. E., et al., J. Biol. Chem. 274:15975-15977 (1999). In vitro polymerization using S. cerevisiae Pol η is disclosed in Washington, M. T., et al., Proc. Natl. Acad. Sci. USA 97:3094-3099 (2000); Johnson, R. E., et al., J. Biol. Chem. 274:15975-15977 (1999). In vitro fidelity assays using S. cerevisiae Pol η are disclosed in Washington, M. T., et al., Proc. Natl. Acad. Sci. USA 97:3094-3099 (2000); Washington, M. T., et al., J. Biol. Chem. 274:36835-36838 (1999). S. cerevisiae Pol η mutants lacking activity are disclosed in Johnson, R. E., et al., J. Biol. Chem. 274:15975-15977 (1999).

The mouse Pol η (XPV) sequences are disclosed in Yamada, A., et al., Nuc. Acids Res. 28:2473-2480 (2000); and GenBank no. AB027128. Expression/purification of mouse Pol η are disclosed in Yamada, A., et al., Nuc. Acids Res. 28:2473-2480 (2000). In vitro polymerization using mouse Pol is disclosed in Yamada, A., et al., Nuc. Acids Res. 28:2473-2480 (2000).

The human Pol η (POLII, also referred to as Rad30A/XPV) sequences are disclosed in Masutani, C., et al., Nature 399:700-704 (1999); Johnson, R. E., et al., Science 285:263-265 (1999); GenBank nos. AB024313 and AF158185. Expression/purification of human Pol η are disclosed in Masutani, C., et al., Nature 399:700-704 (1999); Johnson, R. E., et al., J. Biol. Chem. 275:7447-7450 (2000). Conditions for vitro polymerization using human Pol η are disclosed in Masutani, C., et al., Nature 399:700-704 (1999); Matsuda, T., et al., Nature 404:1011-1013 (2000); Johnson, R. E., et al., J. Biol. Chem. 275:7447-7450 (2000). In vitro fidelity assay using human Pol η are disclosed in Matsuda, T., et al., Nature 404:1011-1013 (2000); Johnson, R. E., et al., J. Biol. Chem. 275:7447-7450 (2000); Bebenek, K., et al., J. Biol. Chem. 276:2317-2320 (2001).

The mouse Pol ι (Rad30b) sequences are disclosed in McDonald, J. P., et al., Genomics 60:20-30 (1999) and GenBank accession no. AF151691.

The human Pol ι (POLI, also referred to as Rad30B) sequences are disclosed in McDonald, J. P., et al., Genomics 60:20-30 (1999) and GenBank no. AF140501. Expression/purification of human Pol t are disclosed in Tissier, A., et al., Gen. Dev. 14:1642-1650 (2000); Zhang, Y., et al., Mol. Cell. Biol. 20:7099-7108 (2000). Conditions for in vitro polymerization using human Pol ι are disclosed in Zhang, Y., et al., Mol. Cell. Biol. 20:7099-7108 (2000). In vitro fidelity assays using human Pol ι are disclosed in Tissier, A., et al., Gen. Dev. 14:1642-1650 (2000); Zhang, Y., et al., Mol. Cell. Biol. 20:7099-7108 (2000). A mutant human Pol ι lacking polymerase activity is disclosed in Tissier, A., et al., Gen. Dev. 14:1642-1650 (2000).

The Translesion DNA polymerases for use in the methods of the invention may be distributive, non processive, or processive.

E. coli PolIII (a superfamily A polymerase) and accessory protein purification (such as β,γ-complex) are disclosed in Naktinis et al., Cell 84:137-145 (1996); Cull, M. G. and McHenry, C. S., Methods Enzymol. 262:22-35 (1995). SSB is available from Amersham-Pharmacia or can be purified as disclosed in Lohman, T. M. and Overman, L. B., J. Biol. Chem. 260:3594-3603 (1985). RecA is available from USB or can be purified as disclosed in Reuven, N. B., et al., J. Biol. Chem. 274:31763-31766 (1999) and Cox, M. M., et al., J. Biol. Chem. 256:4676-4678 (1981).

As mentioned above, non-translesion DNA polymerases may be used to lower the overall mutation rate when combined with a Translesion DNA polymerase. Thus, a combination of one or more non-translesion DNA polymerase and Translesion DNA polymerase may be used in the present methods. A variety of non-translesion DNA polymerases may be used. Such polymerases include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™) DNA polymerase, Pyrococcus furiosus (Pfu) DNA polymerase, DEEPVENT™ DNA polymerase, Pyrococcus woosii (Pwo) DNA polymerase, Pyrococcus sp KDD2 (KOD) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, mycobacterium DNA polymerase (Mtb, Mlep), and mutants, variants and derivatives thereof. RNA polymerases such as T3, T5 and SP6 and mutants, variants and derivatives thereof may also be used in accordance with the invention. Non-translesion DNA polymerases of the invention may be distributive, non processive, or processive.

The non-translesion DNA polymerases used in the present invention may be mesophilic or thermophilic/thermostable. Preferred mesophilic non-translesion DNA polymerases include T7 DNA polymerase, T5 DNA polymerase, Klenow fragment DNA polymerase, DNA polymerase III and the like. Preferred thermostable non-translesion DNA polymerases that may be used in the methods and compositions of the invention include Taq, Tne, Tma, Pfu, Tfl, Tth, Stoffel fragment, VENT™ and DEEPVENT™ DNA polymerases, and mutants, variants and derivatives thereof (U.S. Pat. No. 5,436,149; U.S. Pat. No. 4,889,818; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nucl. Acids Res. 22:3259-3260 (1994)).

Reverse transcriptases for use in this invention include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, R. K., et al, Science 239:487-491 (1988); U.S. Pat. Nos. 4,889,818 and 4,965,188), Tne DNA polymerase (WO 96/10640 and WO 97/09451), Tma DNA polymerase (U.S. Pat. No. 5,374,553) and mutants, variants or derivatives thereof (see, e.g., WO 97/09451 and WO 98/47912). Preferred enzymes for use in the invention include those that have reduced, substantially reduced or eliminated RNase H activity. By an enzyme “substantially reduced in RNase H activity” is meant that the enzyme has less than about 20%, more preferably less than about 15%, 10% or 5%, and most preferably less than about 2%, of the RNase H activity of the corresponding wildtype or RNase H+ enzyme such as wildtype Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, in Kotewicz, M. L., et al, Nucl. Acids Res. 16:265 (1988) and in Gerard, G. F., et al., FOCUS 14(5):91 (1992), the disclosures of all of which are fully incorporated herein by reference. Particularly preferred polypeptides for use in the invention include, but are not limited to, M-MLV reverse transcriptase, RSV reverse transcriptase, AMV reverse transcriptase, RAV (rous-associated virus) reverse transcriptase, MAV (myeloblastosis-associated virus) reverse transcriptase and HIV reverse transcriptase, any of which may be RNase H minus (RNase H−) (see U.S. Pat. No. 5,244,797 and WO 98/47912). It will be understood by one of ordinary skill, however, that any enzyme capable of producing a DNA molecule from a ribonucleic acid molecule (i.e., having reverse transcriptase activity) may be equivalently used in the compositions, methods and kits of the invention.

The non-translesion DNA polymerase may be exonuclease minus (exo⁻) (i.e., lacks proofreading 3′→+5′ and/or 5′→3′ exonuclease activity), substantially reduced in exonuclease activity or exonuclease plus (exo⁺). In the random mutagenesis methods, an exo+non-translesion DNA polymerase is preferred in combination with a Translesion DNA polymerase. For amplification of long nucleic acid molecules (e.g., nucleic acid molecules longer than about 3-5 Kb in length), at least two DNA polymerases (one substantially lacking 3′ exonuclease activity and the other having 3′ exonuclease activity) are typically used. See U.S. Pat. No. 5,436,149; U.S. Pat. No. 5,512,462; Barnes, W. M., Gene 112:29-35 (1992); and WO 98/06736, the disclosures of which are incorporated herein in their entireties. Examples of DNA polymerases substantially lacking in 3′ exonuclease activity include, but are not limited to, Taq, Tne (exo⁻), Tina (exo⁻), Pfu (exo⁻), Pwo (exo) and Tth DNA polymerases, and mutants, variants and derivatives thereof.

The non-translesion DNA polymerases used in the present invention may be isolated from natural or recombinant sources, by techniques that are well-known in the art (See Bej and Mahbubani, Id.; WO 92/06200; WO 96/10640), from a variety of cell lines and organisms that are available commercially (for example, from American Type Culture Collection, Manassass, Va.) or may be obtained by recombinant DNA techniques (WO 96/10640). Suitable for use as sources of thermostable enzymes or the genes thereof for expression in recombinant systems are the thermophilic bacteria Thermus thermophilus, Thermococcus litoralis, Pyrococcus furiosus, Pyrococcus woosii and other species of the Pyrococcus genus, Bacillus sterothermophilus, Sulfolobus acidocaldarius, Thermoplasma acidophilum, Thermus flavus, Thermus ruber, Thermus brockianus, Thermotoga neapolitana, Thermotoga maritima and other species of the Thermotoga genus, and Methanobacterium thermoautotrophicum, and mutants thereof. It is to be understood, however, that thermostable enzymes from other organisms may also be used in the present invention without departing from the scope or preferred embodiments thereof. As an alternative to isolation, thermostable enzymes (e.g., DNA polymerases) are available commercially from, for example, Invitrogen Corporation, New England Biolabs, Finnzymes Oy and Perkin Elmer Cetus.

Preferred non-translesion DNA polymerases in the present invention are T7 DNA Polymerase, T4 DNA Polymerase, E. coli DNA Polymerase I, Klenow Fragment DNA Polymerase, and Tne DNA Polymerase.

The ratio of Translesion DNA polymerase to non-translesion DNA polymerase may be from 10:1 to 1:10, more specifically, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and 1:10.

The present inventions also call for the exclusion of one or more particular non-translesion DNA polymerases. For example, one method, composition or kit may comprise one or more Translesion DNA polymerase and one or more non-translesion polymerase, wherein the non-translesion DNA polymerase is not E. coli DNA Polymerase Pol I, or Klenow fragment of DNA polymerase. The invention may call for the combination of at least one Translesion DNA polymerase and at least one non-translesion DNA polymerase, selected from the group consisting of: (i) E. coli Pol V, wherein said non-translesion DNA polymerase is not E. coli Pol III core, (ii) E. coli Pol V, wherein said non-translesion DNA polymerase is not E. coli Pol III holoenzyme, and (iii) E. coli Pol IV, wherein said non-translesion DNA polymerase is not Klenow fragment. Other non-translesion DNA polymerases which may be excluded from the present methods, compositions and kits include any described above or known in the art. In some embodiments, the at least one Translesion DNA polymerase and at least one non-translesion DNA polymerase will be from different hosts, cells, or cell lines, such as at least one Translesion DNA polymerase from E. coli, and at least one non-translesion DNA polymerase from a host other than E. coli, for example, at least one non-translesion DNA polymerase from yeast or human or mouse. In preferred embodiments, E. coli Pol V or E. coli Pol IV are used with at least one non-translesion DNA polymerase other than E. coli Pol III core, E. coli Pol III holoenzyme, or Klenow fragment. E. coli Pol V or E. coli Pol IV may also be used in combination with at least one other Translesion DNA polymerase and with E. coli Pol III core, E. coli Pol III holoenzyme, or Klenow fragment.

The present methods are preferably carried out in aqueous solutions, preferably comprising one or more buffers and cofactors. Particularly preferred buffers for use in the present methods are the acetate, sulfate, hydrochloride, phosphate or free acid forms of Tris-(hydroxymethyl)aminomethane (TRIS®), although alternative buffers of the same approximate ionic strength and pKa as TRIS® may be used with equivalent results. In addition to the buffer salts, cofactor salts such as those of potassium (preferably potassium chloride or potassium acetate) and magnesium (preferably magnesium chloride or magnesium acetate) are included in the solutions.

In another aspect, the invention includes compositions comprising at least one Translesion DNA polymerase and further comprising at least one component selected from the group consisting of: one or more non-translesion DNA polymerases, one or more reverse transcriptases, one or more nucleotides, one or more buffers, one or more primers, and one or more nucleic acid molecules. The compositions include aqueous solutions as described above, and preferably include one or more buffers as described above.

To form compositions for the present invention, one or more Translesion DNA polymerases are preferably admixed in a buffered salt solution. The compositions may also comprise one or more non-translesion DNA polymerases, which may be an exo+ or an exo− polymerase. One or more nucleotides may optionally be added to make the compositions of the invention. Optionally, one or more of the nucleotides may be modified with one or more modifications, such as with a fluorescent label, which may be same or different modifications. The compositions of the invention may also comprise one or more nucleic acid templates and/or one or more primers. More preferably, the DNA polymerases are provided at working concentrations in stable buffered salt solutions. The terms “stable” and “stability” as used herein generally mean the retention by a composition, such as an enzyme composition, of at least 70%, preferably at least 80%, and most preferably at least 90%, of the original enzymatic activity (in units) after the enzyme or composition containing the enzyme has been stored for about one week at a temperature of about 4° C., about two to six months at a temperature of about −20° C., and about six months or longer at a temperature of about −80° C. As used herein, the term “working concentration” means the concentration of an enzyme that is at or near the optimal concentration used in a solution to perform a particular function (such as reverse transcription of nucleic acids).

The water used in forming the compositions for the present invention is preferably distilled, deionized and sterile filtered (through a 0.1-0.2 micrometer filter), and is free of contamination by DNase and RNase enzymes. Such water is available commercially, for example from Sigma Chemical Company (Saint Louis, Mo.), or may be made as needed according to methods well known to those skilled in the art.

In addition to the enzyme components, compositions for the present invention preferably comprise one or more buffers and cofactors necessary for synthesis of a nucleic acid molecule such as a cDNA molecule. Particularly preferred buffers for use in forming the present compositions are the acetate, sulfate, hydrochloride, phosphate or free acid forms of Tris-(hydroxymethyl)aminomethane (TRIS®), although alternative buffers of the same approximate ionic strength and pKa as TRIS® may be used with equivalent results. In addition to the buffer salts, cofactor salts such as those of potassium (preferably potassium chloride or potassium acetate) and magnesium (preferably magnesium chloride or magnesium acetate) are included in the compositions. Addition of one or more carbohydrates and/or sugars to the compositions and/or synthesis reaction mixtures may also be advantageous, to support enhanced stability of the compositions and/or reaction mixtures upon storage. Preferred such carbohydrates or sugars for inclusion in the compositions and/or synthesis reaction mixtures of the invention include, but are not limited to, sucrose, trehalose, and the like. Furthermore, such carbohydrates and/or sugars may be added to the storage buffers for the enzymes used in the production of the enzyme compositions and kits of the invention. Such carbohydrates and/or sugars are commercially available from a number of sources, including Sigma (St. Louis, Mo.). Compositions for stabilizing DNA polymerases and other enzymes are disclosed in WO 98/06736.

It is often preferable to first dissolve the buffer salts, cofactor salts and carbohydrates or sugars at working concentrations in water and to adjust the pH of the solution prior to addition of the enzymes. In this way, pH-sensitive enzymes will be less subject to acid- or alkaline-mediated inactivation during formulation of the present compositions.

To formulate the buffered salts solution, a buffer salt which is preferably a salt of Tris(hydroxymethyl)aminomethane (TRIS®), and most preferably the hydrochloride salt thereof, is combined with a sufficient quantity of water to yield a solution having a TRIS® concentration of 5-150 millimolar, preferably 10-60 millimolar, and most preferably about 20-60 millimolar. To this solution, a salt of magnesium (preferably either the chloride or acetate salt thereof) may be added to provide a working concentration thereof of 1-10 millimolar, preferably 1.5-8.0 millimolar, and most preferably about 3-7.5 millimolar. A salt of potassium (most preferably potassium chloride) may also be added to the solution, at a working concentration of 10-100 millimolar and most preferably about 20-80 millimolar. A reducing agent such as dithiothreitol may be added to the solution, preferably at a final concentration of about 0.1-20 mM, more preferably a concentration of about 0.5-10 mM, and most preferably at a concentration of about 1 mM. A small amount of a salt of ethylenediaminetetraacetate (EDTA), such as disodium EDTA, may also be added (preferably about 0.1 millimolar). After addition of all buffers and salts, this buffered salt solution is mixed well until all salts are dissolved, and the pH is adjusted using methods known in the art to a pH value of 7.0 to 9.0, preferably 7.5 to 8.5, and most preferably about 8.0.

Polymerases are preferably used in the present methods at a final concentration in a reaction mixture of about 1-10,000 units per milliliter, about 5-5000 units per milliliter, about 10-4000 units per milliliter, about 20-3000 units per milliliter, about 30-3000 units per milliliter, about 40-2000 units per milliliter and most preferably at a concentration of about 50-1000 units per milliliter. Of course, other suitable concentrations of such polymerases suitable for use in the invention will be apparent to one or ordinary skill in the art.

### Sources of Nucleic Acid Template Molecules

Using methods well known in the art, nucleic acid molecules may be prepared from a variety of sources. Preferred nucleic acid molecules for use as templates in the present invention include single-stranded or double-stranded nucleic acid molecule. Such nucleic acid molecules may be derived from natural or non-natural sources including single-stranded or double stranded RNA such as polyadenylated RNA (polyA+ RNA), messenger RNA (mRNA), transfer RNA (tRNA) and ribosomal RNA (rRNA) molecules, genomic DNA, plasmid DNA, or may be synthetic. Nucleic acid templates used in the methods of the invention may comprise one or more genes, partial genes or gene fragments or any number of open reading frames (orfs).

The nucleic acid template molecules that are used to prepare mutagenized or modified molecules according to the methods of the present invention may be prepared synthetically according to standard organic chemical synthesis methods that will be familiar to one of ordinary skill. The nucleic acid template molecules may be obtained from natural sources, such as a variety of cells, tissues, organs or organisms. Cells that may be used as sources of nucleic acid molecules may be prokaryotic (bacterial cells, including those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces) or eukaryotic (including fungi (especially yeasts), plants, protozoans and other parasites, and animals including insects (particularly Drosophila spp. cells), nematodes (particularly Caenorhabditis elegans cells), and mammals (particularly human cells)).

Mammalian somatic cells that may be used as sources of nucleic acids include blood cells (reticulocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, Schwann cells). Mammalian germ cells (spermatocytes and oocytes) may also be used as sources of nucleic acids for use in the invention, as may the progenitors, precursors and stem cells that give rise to the above somatic and germ cells. Also suitable for use as nucleic acid sources are mammalian tissues or organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus.

Any of the above prokaryotic or eukaryotic cells, tissues and organs may be normal, diseased, transformed, established, progenitors, precursors, fetal or embryonic. Diseased cells may, for example, include those involved in infectious diseases (caused by bacteria, fungi or yeast, viruses (including AIDS) or parasites), in genetic or biochemical pathologies (e.g., cystic fibrosis, hemophilia, Alzheimer's disease, muscular dystrophy or multiple sclerosis) or in cancerous processes. Transformed or established animal cell lines may include, for example, COS cells, CHO cells, VERO cells, BHK cells, HeLa cells, HepG2 cells, K562 cells, F9 cells and the like. Other cells, cell lines, tissues, organs and organisms suitable as sources of nucleic acids for use in the present invention will be apparent to one of ordinary skill in the art.

Once the starting cells, tissues, organs or other samples are obtained, nucleic acid molecules (such as mRNA) may be isolated therefrom by methods that are well-known in the art (see, e.g., Maniatis, T., et al., Cell 15:687-701 (1978); Okayama, H., and Berg, P., Mol. Cell. Biol. 2:161-170 (1982); Gubler, U., and Hoffman, B. J., Gene 25:263-269 (1983)). cDNA may be prepared using well-known methods such as those disclosed in WO 98/47912. Nucleic acid molecules may be cloned into vectors such as plasmids or phage (e.g., M13), and vector DNA containing the insert nucleic acid molecule may be purified using standard techniques (see, e.g., J. Sambrook, Molecular Cloning: A Laboratory Manual, Cold Spring Laboratory Press (1989)). In preferred embodiments, the Gene Trapper™ system (Invitrogen Corporation) is used (see, e.g., U.S. Pat. Nos. 5,759,778 and 5,500,356).

General methods for amplification and analysis of nucleic acid molecules or fragments are well-known to one of ordinary skill in the art (see, e.g., U.S. Pat. Nos. 4,683,195; 4,683,202; and 4,800,159; Innis, M. A., et al., eds., PCR Protocols: A Guide to Methods and Applications, San Diego, Calif.: Academic Press, Inc. (1990); Griffin, H. G., and Griffin, A. M., eds., PCR Technology: Current Innovations, Boca Raton, Fla.: CRC Press (1994); PCR Technology: Principles and Applications for DNA Amplification ed. H A Erlich, Stockton Press, New York, N.Y. (1989); PCR Protocols: A Guide to Methods and Applications, eds. Innis, Gelfland, Snisky, and White, Academic Press, San Diego, Calif. (1990); Mattila et al., Nucleic Acids Res. 19: 4967 (1991); Eckert, K. A. and Kunkel, T. A., PCR Methods and Applications 1:17 (1991)). For example, amplification methods include PCR (U.S. Pat. Nos. 4,683,195 and 4,683,202), Strand Displacement Amplification (SDA; U.S. Pat. No. 5,455,166; EP 0 684 315), and Nucleic Acid Sequence-Based Amplification (NASBA; U.S. Pat. No. 5,409,818; EP 0 329 822). Oligonucleotides can be synthesized on an Applied Bio Systems oligonucleotide synthesizer according to specifications provided by the manufacturer.

Typically, the methods of the invention are carried out using one nucleic acid template. For example, the template may be a previously isolated nucleic acid molecule encoding an industrial enzyme. However, the mutagenesis methods of the invention may also be carried out using more than one nucleic acid template, such as a library or population of nucleic acids. Likewise, the methods for synthesizing a modified nucleic acid molecule may use one or more nucleic acid templates, such as a previously isolated clone or a library of clones. Previously isolated nucleic acids may be amplified from sources such as those above using standard techniques and cloned into a suitable vector for use as template in the present methods. Previously isolated nucleic acids may also be subcloned into a suitable vector using standard restriction endonuclease techniques. Template is preferrably single-stranded for use with a mesophilic Translesion DNA polymerase. A preferred method of creating single-stranded template is the GeneTrapper™ system (Invitrogen Corporation) for nucleic acids cloned in a vector containing an F1 origin of replication. Of course, other techniques of nucleic acid synthesis for preparing single or double-stranded template for use in the present methods will be readily apparent to one of ordinary skill in the art.

As discussed, the invention provides methods of incorporating one or more random mutations into a nucleic acid template and also provides methods of synthesizing modified nucleic acid molecules. To carry out the methods of invention, DNA amplification or synthesis is carried out using at least one Translesion DNA polymerase and one or more template nucleic acid molecules. The amplification or synthesis may be one or several rounds. For example, the reverse transcription and mutagenesis reactions may be carried out simultaneously (i.e., in one step) or may be carried our sequentially (i.e., two steps). For methods using mesophilic Translesion DNA polymerases, a single round of amplification or synthesis is preferrably used. In random mutagenesis methods, mutagenized nucleic acids thus produced may optionally be amplified further by standard PCR using any thermophilic Translesion DNA polymerase or thermophilic non-translesion DNA polymerase, as described more fully below.

Polymerase chain reaction (PCR), a well known DNA amplification technique, is a process by which DNA polymerase and deoxyribonucleoside triphosphates are used to amplify a target DNA template. In such PCR reactions, two primers, one complementary to the 3′ termini (or near the 3′-termini) of the first strand of the DNA molecule to be amplified, and a second primer complementary to the 3′ termini (or near the 3′-termini) of the second strand of the DNA molecule to be amplified, are hybridized to their respective DNA molecules. After hybridization, DNA polymerase, in the presence of deoxyribonucleoside triphosphates, allows the synthesis of a third DNA molecule complementary to the first strand and a fourth DNA molecule complementary to the second strand of the DNA molecule to be amplified. This synthesis results in two double stranded DNA molecules. Such double stranded DNA molecules may then be used as DNA templates for synthesis of additional DNA molecules by providing a DNA polymerase, primers, and deoxyribonucleoside triphosphates. As is well known, the additional synthesis is carried out by “cycling” the original reaction (with excess primers and deoxyribonucleoside triphosphates) allowing multiple denaturing and synthesis steps. Typically, denaturing of double stranded DNA molecules to form single stranded DNA templates is accomplished by high temperatures, although it may be accomplished by applying voltage or by other means (see, e.g., U.S. Pat. No. 6,197,508). The thermophilic DNA polymerases (both Translesion DNA polymerases and non-translesion DNA polymerases) used in the present methods are heat stable, and thus will survive such thermal cycling during DNA amplification reactions.

For amplification of long nucleic acid molecules (i.e., greater than about 3-5 Kb in length), the compositions of the invention may comprise a combination of polypeptides having DNA polymerase activity, as described in detail in commonly owned, co-pending U.S. application Ser. No. 08/801,720, filed Feb. 14, 1997, the disclosure of which is incorporated herein by reference in its entirety.

Amplification or synthesis for the methods of the invention may comprise one or more steps. For example, the invention provides a method for random mutagenesis comprising (a) mixing at least one nucleic acid template with one or more of the above-described Translesion DNA polymerases to form a mixture; and (b) incubating the mixture under conditions sufficient to amplify or synthesize or produce one or more nucleic acid molecules complementary to all or a portion of said at least one template. The invention also provides a method for modifying a nucleic acid comprising (a) mixing at least one nucleic acid template with one or more of the above-described Translesion DNA polymerases and one or more modified nucleotides to form a mixture; and (b) incubating the mixture under conditions sufficient to amplify or synthesize or produce one or more nucleic acid molecules complementary to all or a portion of said at least one template.

For methods using more than one Translesion DNA polymerase, the enzymes may be used simultaneously or sequentially. For methods using one or more thermophilic Translesion DNA polymerases and one or more thermophilic non-translesion DNA polymerases, the enzymes may be mixed with the template prior to cycling.

For methods using one or more mesophilic Translesion DNA polymerases and one or more thermophilic non-translesion DNA polymerases, the enzymes may be added simultaneously or sequentially. For example, the mesophilic and thermophilic enzymes may be mixed with the template simultaneously, the first round of amplification carried out at a moderate temperature (such as less then 40° C.), and the subsequent rounds of PCR reactions carried out by thermal cycling. Alternatively, the mesophilic enzyme is mixed with the template, the first round of amplification is carried out at a moderate temperature, after which the thermophilic enzyme is added, and subsequent rounds of amplification are then carried out.

The invention also provides nucleic acid molecules mutagenized or modified by such methods. The invention further provides host cells comprising the present mutagenized nucleic acid molecules, and polypeptides encoded by the present mutagenized nucleic acid molecules.

Modified nucleic acid molecules produced by the present methods may be purified or may be used directly to detect or analyze nucleic acids of interest by above-mentioned methods and other methods well known in the art.

The present random mutagenesis methods produce a population of mutagenized nucleic acids, which may be isolated for further characterization and use. This may be accomplished by separation of the nucleic acid by size or by any physical or biochemical means including gel electrophoresis, capillary electrophoresis, chromatography (including sizing, affinity and immunochromatography), density gradient centrifugation and immunoadsorption, optionally after endonuclease digestion, PCR amplification, or other enzymatic manipulation. Separation of nucleic acids by gel electrophoresis is particularly preferred, as it provides a rapid and highly reproducible means of sensitive separation of a multitude of nucleic acid fragments, and permits direct, simultaneous comparison of the fragments in several samples of nucleic acids.

The isolated unique nucleic acid fragments or generally any of the nucleic acid molecules of the invention may be inserted into standard vectors, including expression vectors, suitable for transfection or transformation of a variety of prokaryotic (bacterial) or eukaryotic (yeast, plant or animal including human and other mammalian) cells. Alternatively, nucleic acid molecules that are mutagenized using the methods of the present invention may be further characterized, for example by sequencing (i.e., determining the nucleotide sequence of the nucleic acid fragments), by methods described below and others that are standard in the art (see, e.g., U.S. Pat. Nos. 4,962,022 and 5,498,523, which are directed to methods of DNA sequencing).

After cloning, the mutangenized nucleic acids are then screened to identify individuals encoding proteins or polypeptides having new or altered activities such as enzymatic activities, stability, ligand-binding, receptor-binding, antigen-binding affinity, therapeutic efficacy, teratogenicity, etc. The selection of an assay will be dictated by the activity being screened and will be apparent to the artisan of ordinary skill. For example, ELISAs may be performed to assay for antibody-binding activity. Once a mutagenized nucleic acid is identified that encodes a new or altered gene product that exhibits the desired activity, it may be isolated for further characterization or use.

### Vectors and Host Cells

The present invention also relates to vectors which comprise the isolated nucleic acid molecules of the present invention, host cells which are genetically engineered with the recombinant vectors, and methods for the production of a recombinant polypeptide using these vectors and host cells.

The vector used in the present invention may be, for example, a phage or a plasmid, and is preferably a plasmid. Preferred are vectors comprising cis-acting control regions to the nucleic acid encoding the polypeptide of interest. Appropriate trans-acting factors may be supplied by the host, supplied by a complementing vector or supplied by the vector itself upon introduction into the host.

In certain preferred embodiments in this regard, the vectors provide for specific expression of a polypeptide encoded by the nucleic acid molecules of the invention; such expression vectors may be inducible and/or cell type-specific. Particularly preferred among such vectors are those inducible by environmental factors that are easy to manipulate, such as temperature and nutrient additives.

Expression vectors useful in the present invention include chromosomal-, episomal-and virus-derived vectors, e.g., vectors derived from bacterial plasmids or bacteriophages, and vectors derived from combinations thereof, such as cosmids and phagemids.

The nucleic acid insert should be operatively linked to an appropriate promoter, such as the phage lambda P_{L} promoter, the E. coli lac, trp and tac promoters. Other suitable promoters will be known to the skilled artisan. The gene fusion constructs will further contain sites for transcription initiation, termination and, in the transcribed region, a ribosome binding site for translation. The coding portion of the mature transcripts expressed by the constructs will preferably include a translation initiation codon at the beginning, and a termination codon (UAA, UGA or UAG) appropriately positioned at the end, of the polynucleotide to be translated.

The expression vectors will preferably include at least one selectable marker. Such markers include tetracycline or ampicillin resistance genes for culturing in E. coli and other bacteria.

Among vectors preferred for use in the present invention include pQE70, pQE60 and pQE-9, available from Qiagen; pBS vectors, Phagescript vectors, Bluescript vectors, pNH8A, pNH16a, pNH18A, pNH46A, available from Stratagene; pcDNA3 available from Invitrogen Corporation; and pGEX, pTrxfus, pTrc99 a, pET-5, pET-9, pKK223-3, pKK233-3, pDR540, pRIT5 available from Pharmacia. Other suitable vectors will be readily apparent to the skilled artisan.

Representative examples of appropriate host cells include, but are not limited to, bacterial cells such as E. coli, Streptomyces spp., Erwinia spp., Klebsiella spp. and Salmonella typhimurium. Preferred as a host cell is E. coli, and particularly preferred are E. coli strains DH10B and Stb12, which are available commercially (Life Technologies Division of Invitrogen Corporation, Rockville, Md.).

Additional expression vectors and host cells may be preferred for screening mutagenized nucleic acids and their encoded proteins for particular new or altered activites. Such expression vectors and host cells will be apparent to the artisan of ordinary skill.

### Peptide Production

As noted above, the methods of the present invention are suitable for production of any polypeptide of any length, via insertion of the above-described nucleic acid molecules or vectors into a host cell and expression of the nucleotide sequence encoding the polypeptide of interest by the host cell. Introduction of the nucleic acid molecules or vectors into a host cell to produce a transformed host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986). Expression of polypeptides encoded by the nucleic acid molecules of the invention may also be accomplished by in vitro transcription/translation systems.

Once transformed host cells have been obtained, the cells may be cultivated under any physiologically compatible conditions of pH and temperature, in any suitable nutrient medium containing assimilable sources of carbon, nitrogen and essential minerals that support host cell growth. Recombinant polypeptide-producing cultivation conditions will vary according to the type of vector used to transform the host cells. For example, certain expression vectors comprise regulatory regions which require cell growth at certain temperatures, or addition of certain chemicals or inducing agents to the cell growth medium, to initiate the gene expression resulting in the production of the recombinant polypeptide. Thus, the term “recombinant polypeptide-producing conditions,” as used herein, is not meant to be limited to any one set of cultivation conditions. Appropriate culture media and conditions for the above-described host cells and vectors are well-known in the art.

Following its production in the host cells, the polypeptide of interest may be isolated by several techniques. To liberate the polypeptide of interest from the host cells, the cells are lysed or ruptured. This lysis may be accomplished by contacting the cells with a hypotonic solution, by treatment with a cell wall-disrupting enzyme such as lysozyme, by sonication, by treatment with high pressure, or by a combination of the above methods. Other methods of bacterial cell disruption and lysis that are known to one of ordinary skill may also be used.

Following disruption, the polypeptide may be separated from the cellular debris by any technique suitable for separation of particles in complex mixtures. The polypeptide may then be purified by well known isolation techniques. Suitable techniques for purification include, but are not limited to, ammonium sulfate or ethanol precipitation, acid extraction, electrophoresis, immunoadsorption, anion or cation exchange chromatography, phosphocellulose chromatography, hydrophobic interaction chromatography, affinity chromatography, immunoaffinity chromatography, size exclusion chromatography, liquid chromatography (LC), high performance LC(HPLC), fast performance LC (FPLC), hydroxylapatite chromatography and lectin chromatography.

### Kits

The present invention also provides kits for use in the mutagenesis or modification (e.g., labeling) of a nucleic acid molecule. Mutagenesis kits and nucleic acids modification kits according to the present invention comprise a carrier means, such as a box, carton, tube or the like, having in close confinement therein one or more container means, such as vials, tubes, ampules, bottles and the like. In one aspect, a first container means contains a stable composition comprising a mixture of reagents, at working concentrations, which are at least one Translesion DNA polymerase, at least one buffer salt, and at least one deoxynucleoside triphosphate.

For mutagenesis, the kits of the invention may comprise one or more of the following components: (i) one or more Translesion DNA polymerases, (ii) one or more non-translesion DNA polymerase, (iii) one or more suitable buffers, (iv) one or more nucleotides, and (v) one or more primers.

For synthesizing modified nucleic acids, the kits of the invention may comprise one or more of the following components: (i) one or more Translesion DNA polymerases, (ii) one or more non-translesion polymerase, (iii) one or more suitable buffers, (iv) one or more nucleotides, (v) one or more modified nucleotides, and (vi) one or more primers.

The kits may further comprise additional reagents and compounds necessary for carrying out standard nucleic synthesis protocols (Sec U.S. Pat. Nos. 4,683,195 and 4,683,202, which are directed to methods of DNA amplification by PCR; WO 00/71559, directed to methods of producing improved primers, WO 98/06736, directed to stable compositions of DNA polymerases).

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, e.g., J. Sambrook, et al., Molecular Cloning: A Laboratory Manual, Second Edition, Books 1-3, Cold Spring Harbor Laboratory Press (1989); B. Roe, et al., DNA Isolation and Sequencing: Essential Techniques, John Wiley & Sons (1984); J. M. Polak and James O′D. McGee, In Situ Hybridization: Principles and Practice; Oxford University Press (1990); M. J. Gait (Editor), Oligonucleotide Synthesis: A Practical Approach, Irl Press (1996); and, D. M. J. Lilley and J. E. Dahlberg, Methods of Enzymology: DNA Structure Part A: Synthesis and Physical Analysis of DNA Methods in Enzymology, Academic Press (1992).

It will be readily apparent to those of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, public nucleotide and amino acid sequences, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A method for amplifying or synthesizing or producing a nucleic acid molecule comprising:
(a) combining at least one nucleic acid template, at least one Translesion DNA polymerase, and at least one non-translesion DNA polymerase; and
(b) incubating the combination of (a) under conditions sufficient to amplify, synthesize or produce one or more nucleic acid molecules complementary to all or a portion of said at least one template.

2. The method of claim 1, wherein the combination of (a) comprises at least one Translesion DNA polymerase selected from the group consisting of: (i) E. coli Pol V, wherein said non-translesion DNA polymerase is not E. coli Pol III core, (ii) E. coli Pol V, wherein said non-translesion DNA polymerase is not E. coli Pol III holoenzyme, and (iii) E. coli Pol IV, wherein said non-translesion DNA polymerase is not Klenow fragment.

3. The method of claim 1 or claim 2, wherein said at least one Translesion DNA polymerase incorporates at least one mismatch into said complementary nucleic acid molecule.

4. The method of claim 1, wherein said at least one Translesion DNA polymerase incorporates at least one modified nucleotide into said complementary nucleic acid molecule.

5. A method for incorporating a mutation into a nucleic acid molecule comprising:
(a) combining at least one nucleic acid template and at least one Translesion DNA polymerase; and
(b) incubating the combination of (a) under conditions sufficient to produce one or more nucleic acid molecules complementary to all or a portion of said at least one template, wherein said complementary nucleic acid molecule comprises at least one mismatch.

6. The method of claim 5, wherein said method allows incorporation of one or more random mutations into a nucleic acid molecule.

7. The method of claim 5, wherein the combination of (a) comprises at least one Translesion DNA polymerase selected from the group consisting of: mesophilic polymerases and thermophilic polymerases.

8. The method of claim 7, wherein the combination of (a) comprises at least one Translesion DNA polymerase selected from the group consisting of: vertebrate Translesion DNA polymerases, mammalian Translesion DNA polymerases, animal Translesion DNA polymerases, insect Translesion DNA polymerases, bacterial Translesion DNA polymerases, eubacterial Translesion DNA polymerases, and archaebacterial Translesion DNA polymerases.

9. The method of claim 8, wherein the combination of (a) comprises at least one Translesion DNA polymerase selected from the group consisting of: E. coli Translesion DNA polymerases, Sulfolobus sofataricus Translesion DNA polymerases, human Translesion DNA polymerases, mouse Translesion DNA polymerases, and S. cerevisiae Translesion DNA polymerases.

10. The method of claim 9, wherein the combination of (a) comprises at least one Translesion DNA polymerase selected from S. cerevisiae Translesion DNA polymerases.

11. The method of claim 5, wherein the combination of (a) comprises at least one Translesion DNA polymerase selected from the group consisting of: Pol V, Pol IV, Pol κ, Pol η, Pol ι, and Pol ζ.

12. The method of claim 5 or claim 10, wherein the combination of (a) comprises Pol κ and Pol η.

13. The method of claim 5 or claim 10, wherein the combination of (a) comprises Polκ, Polη, and Pol ζ.

14. The method of claim 5 or claim 10, wherein the combination of (a) comprises Pol κ and Pol ζ.

15. The method of claim 5 or claim 10, wherein the combination of (a) comprises Pol η and Pol ζ.

16. The method of claim 5, wherein the combination of (a) comprises Pol V and Pol ζ.

17. The method of claim 5, wherein the combination of (a) further comprises a non-translesion DNA polymerase.

18. The method of claim 17, wherein said template is mRNA or a population of mRNA and said non-translesion DNA polymerase is a reverse transcriptase and said method comprises one step or two steps.

19. The method of claim 17, wherein said non-translesion DNA polymerase has exonuclease activity.

20. The method of claim 19, wherein said non-translesion DNA polymerase is selected from the group consisting of: T7 DNA Polymerase, T4 DNA Polymerase, E. coli DNA Polymerase I, Klenow Fragment DNA Polymerase, and Tne DNA Polymerase.

21. The method of claim 17, wherein said non-translesion DNA polymerase is a non processive DNA polymerase.

22. The method of claim 21, wherein said non-translesion DNA polymerase is a non processive mutant wherein the enzyme is made non processive by point mutation.

23. The method of claim 20, wherein said non-translesion DNA polymerase is Klenow fragment DNA polymerase.

24. The method of claim 22, wherein said non-translesion DNA polymerase is a non processive mutant of Klenow fragment DNA polymerase wherein the enzyme is made non processive by point mutation.

25. The method of claim 5 or claim 10, wherein said Translesion DNA polymerase is non processive or processive.

26. A method for incorporating a mutation into a nucleic acid molecule comprising:
(a) combining at least one nucleic acid template and at least two polymerases selected from the group consisting of: (i) at least one Translesion DNA polymerase and at least one non-translesion DNA polymerase, and (ii) at least two Translesion DNA polymerases; and
(b) incubating the combination of (a) under conditions sufficient to produce a nucleic acid molecule complementary to all or a portion of said at least one template, wherein said complementary nucleic acid molecule comprises at least one mismatch.

27. The method of claim 26, wherein said method allows incorporation of one or more random mutations into a nucleic acid molecule.

28. The method of claim 26, wherein the combination of (a) comprises at least one Translesion DNA polymerase and at least one non-translesion DNA polymerase.

29. The method of claim 28, wherein the combination of (a) comprises at least one Translesion DNA polymerase selected from the group consisting of: mesophilic polymerases and thermophilic polymerases.

30. The method of claim 29, wherein the combination of (a) comprises at least one Translesion DNA polymerases selected from the group consisting of: vertebrate Translesion DNA polymerases, mammalian Translesion DNA polymerases, animal Translesion DNA polymerases, insect Translesion DNA polymerases, bacterial Translesion DNA polymerases, eubacterial Translesion DNA polymerases, and archaebacterial Translesion DNA polymerases.

31. The method of claim 30, wherein the combination of (a) comprises at least one Translesion DNA polymerase selected from the group consisting of: E. coli Translesion DNA polymerases, Sulfolobus sofataricus Translesion DNA polymerases, human Translesion DNA polymerases, mouse Translesion DNA polymerases, and S. cerevisiae Translesion DNA polymerases.

32. The method of claim 31, wherein the combination of (a) comprises at least one Translesion DNA polymerase selected from S. cerevisiae Translesion DNA polymerases.

33. The method of claim 26, wherein the combination of (a) comprises at least one Translesion DNA polymerase selected from the group consisting of: Pol V, Pol IV, Pol κ, Pol η, Pol ι, and Pol ζ.

34. The method of claim 26 or claim 32, wherein the combination of (a) comprises Pol κ and Pol η.

35. The method of claim 26 or claim 32, wherein the combination of (a) comprises Pol κ, Pol η, and Pol ζ.

36. The method of claim 26 or claim 32, wherein the combination of (a) comprises Pol η and Pol ζ.

37. The method of claim 26 or claim 32, wherein the combination of (a) comprises Pol it and Pol ζ.

38. The method of claim 26, wherein the combination of (a) comprises Pol V and Pol C.

39. The method of claim 27, wherein said template is mRNA or a population of mRNA and said non-translesion DNA polymerase is a reverse transcriptase and said method comprises one step or two steps.

40. The method of claim 26, wherein said at least one non-translesion DNA polymerase has exonuclease activity.

41. The method of claim 40, wherein said non-translesion DNA polymerase is selected from the group consisting of: T7 DNA Polymerase, T4 DNA Polymerase, E. coli DNA Polymerase I, Klenow Fragment DNA Polymerase, and Tne DNA Polymerase.

42. The method of claim 28, wherein said non-translesion DNA polymerase is a non processive DNA polymerase.

43. The method of claim 42, wherein said non-translesion DNA polymerase is a non processive mutant wherein the enzyme is made non processive by point mutation.

44. The method of claim 41, wherein said non-translesion DNA polymerase is Klenow fragment DNA polymerase.

45. The method of claim 43, wherein said non-translesion DNA polymerase is a non processive mutant of Klenow fragment DNA polymerase wherein the enzyme is made non processive by point mutation.

46. The method of claim 28, wherein said Translesion DNA polymerase is non processive or processive.

47. The method of claim 26, wherein the combination of (a) comprises at least two Translesion DNA polymerases.

48. The method of claim 47, wherein the combination of (a) comprises at least two Translesion DNA polymerases selected from the group consisting of: mesophilic polymerases and thermophilic polymerases.

49. The method of claim 48, wherein the combination of (a) comprises at least two Translesion DNA polymerases selected from the group consisting of: vertebrate Translesion DNA polymerases, mammalian Translesion DNA polymerases, animal Translesion DNA polymerases, insect Translesion DNA polymerases, bacterial Translesion DNA polymerases, eubacterial Translesion DNA polymerases, and archaebacterial Translesion DNA polymerases.

50. The method of claim 47, wherein the combination of (a) comprises at least two Translesion DNA polymerase selected from the group consisting of: E. coli Translesion DNA polymerases, Sulfolobus sofataricus Translesion DNA polymerases, human Translesion DNA polymerases, mouse Translesion DNA polymerases, and S. cerevisiae Translesion DNA polymerases.

51. The method of claim 50, wherein the combination of (a) comprises at least one Translesion DNA polymerase selected from S. cerevisiae Translesion DNA polymerases.

52. The method of claim 47, wherein the combination of (a) comprises at least one Translesion DNA polymerase selected from the group consisting of: Pol V, Pol IV, Pol κ, Pol η, Pol ι, and Pol ζ.

53. The method of claim 47 or claim 52, wherein the combination of (a) comprises Pol κ and Pol η.

54. The method of claim 47 or claim 52, wherein the combination of (a) comprises Pol κ, Pol η, and Pol ζ.

55. The method of claim 47 or claim 52, wherein the combination of (a) comprises Pol κ and Pol ζ.

56. The method of claim 47 or claim 52, wherein the combination of (a) comprises Pol η and Pol ζ.

57. The method of claim 47, wherein the combination of (a) comprises Pol V and Pol ζ.

58. The method of claim 47, wherein said combination of (a) further comprises a non-translesion DNA polymerase having exonuclease activity.

59. The method of claim 47, wherein said template is mRNA or a population of mRNA and said non-translesion DNA polymerase is a reverse transcriptase and said method comprises one step or two steps.

60. The method of claim 58, wherein said non-translesion DNA polymerase is selected from the group consisting of: T7 DNA Polymerase, T4 DNA Polymerase, E. coli DNA Polymerase I, Klenow Fragment DNA Polymerase, and Tne DNA Polymerase.

61. The method of claim 58, wherein said non-translesion DNA polymerase is a non processive DNA polymerase.

62. The method of claim 61, wherein said non-translesion DNA polymerase is a non processive mutant wherein the enzyme is made non processive by point mutation.

63. The method of claim 60, wherein said non-translesion DNA polymerase is Klenow fragment DNA polymerase.

64. The method of claim 62, wherein said non-translesion DNA polymerase is a non processive mutant of Klenow fragment DNA polymerase wherein the enzyme is made non processive by point mutation.

65. The method of claim 47, wherein said Translesion DNA polymerase is non processive or processive

66. A mutagenized nucleic acid molecule produced by the method of any one of claim 3, 5, or 26.

67. A host cell comprising the mutagenized nucleic acid molecule of claim 66.

68. A vector comprising the mutagenized nucleic acid molecule of claim 66.

69. A host cell comprising the vector of claim 68.

70. A method of producing a recombinant host cell comprising introducing the mutagenized nucleic acid molecule of claim 66 into a host cell.

71. A method of producing a mutagenized polypeptide comprising: culturing the host cell of claim 67 and expressing at least one polypeptide encoded by the mutagenized nucleic acid molecule.

72. The method of claim 71, further comprising isolating said at least one polypeptide.

73. A method of producing a mutagenized polypeptide comprising: obtaining a nucleic acid molecule of claim 66 and expressing at least one polypeptide encoded by said nucleic acid molecule.

74. A polypeptide produced by the method any one of claim 71 and 73.

75. A method for incorporating one or more modified nucleotides into a nucleic acid molecule comprising:
(a) combining at least one nucleic acid template, at least one modified nucleotide, and at least one Translesion DNA polymerase; and
(h) incubating the combination of (a) under conditions sufficient to produce one or more nucleic acid molecules complementary to all or a portion of said at least one template, wherein said complementary nucleic acid molecule comprises at least one modified nucleotide.

76. The method of claim 75, wherein the combination of (a) comprises at least one Translesion DNA polymerase selected from the group consisting of: mesophilic polymerases and thermophilic polymerases.

77. The method of claim 76, wherein the combination of (a) comprises at least one Translesion DNA polymerases selected from the group consisting of: vertebrate Translesion DNA polymerases, mammalian Translesion DNA polymerases, animal Translesion DNA polymerases, insect Translesion DNA polymerases, bacterial Translesion DNA polymerases, eubacterial Translesion DNA polymerases, and archaebacterial Translesion DNA polymerases.

78. The method of claim 77, wherein the combination of (a) comprises at least one Translesion DNA polymerase selected from the group consisting of: E. coli Translesion DNA polymerases, Sulfolobus sofataricus Translesion DNA polymerases, human Translesion DNA polymerases, mouse Translesion DNA polymerases, and S. cerevisiae TranslesionDNA polymerases.

79. The method of claim 78, wherein the combination of (a) comprises at least one Translesion DNA polymerase selected from S. cerevisiae Translesion DNA polymerases.

80. The method of claim 75, wherein the combination of (a) comprises at least one Translesion DNA polymerase selected from the group consisting of: Pol V, Pol IV, Pol κ, Pol η, Pol ι, and Pol ζ.

81. The method of claim 75, wherein the combination of (a) comprises Pol ι.

82. The method of claim 75, wherein the combination of (a) comprises Pol η.

83. The method of claim 75, wherein the combination of (a) comprises Pol ι and Pol η.

84. The method of claim 75, wherein said modified nucleotide comprises a label.

85. The method of claim 84, wherein said label is selected from the group consisting of: radioactive labels, metal labels, gold, magnetic resonance labels, dye labels, fluorescent labels, chemiluminescent labels, electrochemiluminescent labels, bioluminescent labels, enzyme labels, antigenic determinants, biotin labels, and digoxigenin labels (DIG).

86. The method of claim 85, wherein said label is a fluorescent label.

87. The method of claim 86, wherein said fluorescent label is selected from the group consisting of: fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) said benzoic acid (DABCYL), Cascade Blue™, Oreg. Green™, Texas Red™, FluoroLink™, Cyanine, and 5-(2′-aminoethyl)aminonaphthalene-1-sulfonic acid or (EDANS).

88. A modified nucleic acid produced by the method of claim 75.

89. A method of detecting the presence or absence of one or more nucleic acids in a sample or determining the amount of one or more nucleic acid molecules in a sample or analyzing one or more nucleic acid molecules in a sample comprising: (a) hybridizing the modified nucleic acid of claim 68 to said one or more nucleic acid molecules, and (b) detecting the presence or absence of one or more nucleic acids or determining the amount of one or more nucleic acid molecules or analyzing one or more nucleic acid molecules.

90. The method of claim 89, wherein said modified nucleic acid allows for said detecting.

91. A kit for incorporating a mutation into one or more nucleic acid molecules comprising at least one Translesion DNA polymerase.

92. The kit of claim 91, further comprising at least one non-translesion DNA polymerase.

93. The kit of claim 92, further comprising one or more components selected from the group consisting of: one or more reverse transcriptase, one or more nucleotides, a suitable buffer, and one or more primers.

94. A kit for incorporating modified nucleotides into one or more nucleic acid molecules comprising at least one Translesion DNA polymerase.

95. The kit of claim 94, further comprising one or more modified nucleotides.

96. The kit of claim 95, further comprising one or more components selected from the group consisting of: one or more nucleotides, a suitable buffer, and one or more primers.

97. A composition comprising at least one Translesion DNA polymerase and further comprising at least one component selected from the group consisting of: one or more non-translesion DNA polymerases, one or more reverse transcriptases, one or more nucleotides, one or more buffers, one or more primers, and one or more nucleic acid molecules.

98. A reaction mixture comprising at least one Translesion DNA polymerase and further comprising at least one component selected from the group consisting of: one or more non-translesion DNA polymerases, one or more reverse transcriptases, one or more nucleotides, one or more buffers, one or more primers, and one or more nucleic acid molecules.
